(19) United States
(12) Patent Application Publication
(10) Pub. No.: US 2025/0140515 A1
(43) Pub. Date: May 1, 2025

(54) X-RAY IMAGING SYSTEM FOR RADIATION THERAPY

(71) Applicant: SHANGHAI UNITED IMAGING HEALTHCARE CO., LTD., Shanghai (CN)

(72) Inventors: Buliang SUN, Shanghai (CN); Cheng NI, Shanghai (CN); Wei ZHANG, Shanghai (CN); Li WANG, Shanghai (CN); Tao LIU, Shanghai (CN); Jianwei FU, Shanghai (CN); Libo FANG, Shanghai (CN); Bo CAI, Shanghai (CN); Xiaohua ZHU, Shanghai (CN); Can LIAO, Shanghai (CN); Hua FAN, Shanghai (CN); Hongcheng YANG, Shanghai (CN); Kang SHEN, Shanghai (CN); Qiudong WANG, Shanghai (CN); Yanfang LIU, Shanghai (CN); Jingjie ZHOU, Shanghai (CN); Kangkang SHA, Shanghai (CN)

(73) Assignee: SHANGHAI UNITED IMAGING HEALTHCARE CO., LTD., Shanghai (CN)

(21) Appl. No.: 19/010,300

(22) Filed: Jan. 6, 2025

Related U.S. Application Data
(63) Continuation-in-part of application No. 18/180,811, filed on Mar. 8, 2023, now Pat. No. 12,186,591.
Application No. 18/180,811 is a continuation of application No. PCT/CN2021/080639, filed on Mar. 12, 2021.
Application No. PCT/CN2021/080639 is a continuation-in-part of application No. 17/015,033, filed on Sep. 8, 2020, now Pat. No. 11,883,687.

(30) Foreign Application Priority Data
Nov. 7, 2020 (CN) 202011234813.9
Nov. 13, 2020 (CN) 202011271345.2
Dec. 14, 2020 (CN) 202011468108.5
Jun. 21, 2024 (CN) 202410817027.3
Jun. 21, 2024 (CN) 202421445992.4

(51) Int. Cl. H01J 37/244; H01J 37/28

(52) U.S. Cl. CPC H01J 37/244; H01J 37/28; H01J 2237/0458; H01J 2237/24475; H01J 2237/2448

(57) ABSTRACT

An imaging device is provided. The imaging device includes: one or more imaging sources; and a plurality of detectors combined by joining, the plurality of detectors being configured to receive one or more imaging beams emitted by the one or more imaging sources.

### CROSS-REFERENCE TO THE RELATED APPLICATIONS

This application claims priority of Chinese Application No. 202421445992.4 filed on Jun. 21, 2024, and Chinese Application No. 202410817027.3 filed on Jun. 21, 2024, and is a continuation in part of U.S. application Ser. No. 18/180,811, filed on Mar. 8, 2023, which is a continuation of International Patent Application No. PCT/CN2021/080639, filed on Mar. 12, 2021, which claims priority of U.S. patent application Ser. No. 17/015,033 (issued as U.S. Pat. No. 11,883,687) filed on Sep. 8, 2020, Chinese Application No. 202011234813.9 filed on Nov. 7, 2020, Chinese Application No. CN202011271345.2 filed on Nov. 13, 2020, and Chinese Application No. CN202011468108.5 filed on Dec. 14, 2020, the contents of each of which are hereby incorporated by reference.

### TECHNICAL FIELD

The present disclosure generally relates to medical technology, and more particularly, systems and methods for imaging systems for radiation therapy.

### BACKGROUND

Radiation therapy is a localized treatment for a specific target tissue (a target volume), such as a cancerous tumor. Dosimetric and geometric data are checked before, after, or during the treatment, to ensure correct patient placement and that the administered radiotherapy treatment matches the previously planned treatment. This process is referred to as image-guided radiation therapy (IGRT), and involves the use of an imaging system to view target tissues while radiation treatment is delivered to the target volume.

There is an increasing demand for multi-modality imaging technologies in the fields such as radiation delivery (e.g., radiation therapy, radiation processing, radiation verification, etc.), medical diagnostics, industrial inspection, etc. For example, radiation therapy (radiotherapy) is a treatment in which a radiation beam passes through a target portion of an object (e.g., a patient) to reach an injury or tumor (e.g., a target region) in the body of the patient. Radiotherapy plays an increasingly important role in tumor treatment. In order to provide more precise irradiation of a tumor site, and better protect critical organs around the tumor site, various imaging means may be applied in IGRT, such as Digital Radiography (DR) imaging, Computed Tomography (CT) imaging, etc. However, the multi-modality imaging system (e.g., a radiotherapy system integrated with DR imaging and CT imaging) designed in practical application may be expensive and difficult to manufacture.

Therefore, it is desirable to provide a multi-modality imaging system with relatively high feasibility and efficiency.

### SUMMARY

According to one aspect of the present disclosure, an imaging device may be provided. The imaging device may include: one or more imaging sources; and a plurality of detectors combined by joining, the plurality of detectors being configured to receive one or more imaging beams emitted by the one or more imaging sources.

According to another aspect of the present disclosure, an imaging device may be provided. The imaging device may include: one or more imaging sources configured to emit at least two different types of imaging beams; and one or more detectors configured to receive the at least two different types of imaging beams to generate images of at least two different modalities or with at least two different energy levels.

According to another aspect of the present disclosure, a radiation system may be provided. The system may include an imaging device configured to scan a target object to generate one or more images of the target object; and a radiation device configured to deliver a radiation beam to the target object based on the one or more images; wherein the imaging device includes: one or more imaging sources; and a plurality of detectors combined by joining, the plurality of detectors being configured to receive one or more imaging beams emitted by the one or more imaging sources.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary radiation system according to some embodiments of the present disclosure;

FIG. 2A is a schematic diagram illustrating an exemplary configuration of a radiation device according to some embodiments of the present disclosure;

FIG. 2B is a schematic diagram illustrating an exemplary imaging beam detector according to some embodiments of the present disclosure;

FIGS. 3-5 are schematic diagrams illustrating exemplary configurations of a radiation device according to some embodiments of the present disclosure;

FIG. 6 is a schematic diagram illustrating exemplary hardware and/or software components of a computing device according to some embodiments of the present disclosure;

FIG. 7 is a schematic diagram illustrating exemplary hardware and/or software components of a mobile device according to some embodiments of the present disclosure;

FIG. 8 is a block diagram illustrating an exemplary processing device according to some embodiments of the present disclosure;

FIG. 9 is a flowchart illustrating an exemplary imaging process of a radiation system according to some embodiments of the present disclosure;

FIG. 10 is a flowchart illustrating an exemplary imaging process of a radiation system according to some embodiments of the present disclosure;

FIG. 11 is a flowchart illustrating an exemplary imaging process of a radiation system according to some embodiments of the present disclosure;

FIG. 12 is a flowchart illustrating an exemplary imaging process of a radiation system according to some embodiments of the present disclosure;

FIG. 13 is a schematic diagram illustrating an exemplary structure of an imaging device according to some embodiments of the present disclosure;

FIG. 14A is a schematic diagram illustrating another exemplary structure of an imaging device according to some embodiments of the present disclosure;

FIG. 14B is a schematic diagram illustrating another exemplary structure of an imaging device according to some embodiments of the present disclosure;

FIG. 15 is a schematic diagram illustrating another exemplary structure of an imaging device according to some embodiments of the present disclosure;

FIG. 16 is a schematic diagram illustrating another exemplary structure of an imaging device according to some embodiments of the present disclosure;

FIG. 17 is a schematic diagram illustrating another exemplary structure of an imaging device according to some embodiments of the present disclosure;

FIG. 18 is a schematic diagram illustrating another exemplary structure of an imaging device according to some embodiments of the present disclosure;

FIG. 19 is a schematic diagram illustrating another exemplary structure of an imaging device according to some embodiments of the present disclosure;

FIG. 20 is a schematic diagram illustrating another exemplary structure of an imaging device according to some embodiments of the present disclosure;

FIG. 21 is a schematic diagram illustrating another exemplary structure of an imaging device according to some embodiments of the present disclosure;

FIG. 22 is a schematic diagram illustrating another exemplary structure of an imaging device according to some embodiments of the present disclosure;

FIG. 23 is a schematic diagram illustrating another exemplary structure of an imaging device according to some embodiments of the present disclosure;

FIG. 24 is a schematic diagram illustrating another exemplary structure of an imaging device according to some embodiments of the present disclosure;

FIG. 25 is a schematic diagram illustrating another exemplary structure of an imaging device according to some embodiments of the present disclosure;

FIG. 26 is a schematic diagram illustrating another exemplary structure of an imaging device according to some embodiments of the present disclosure;

FIG. 27 is a schematic diagram illustrating another exemplary structure of an imaging device according to some embodiments of the present disclosure;

FIG. 28 is a schematic diagram illustrating another exemplary structure of an imaging device according to some embodiments of the present disclosure;

FIG. 29 is a schematic diagram illustrating another exemplary structure of an imaging device according to some embodiments of the present disclosure;

FIG. 30 is a schematic diagram illustrating another exemplary structure of an imaging device according to some embodiments of the present disclosure;

FIG. 31 is a schematic diagram illustrating an exemplary structure of a radiation system according to some embodiments of the present disclosure; and

FIG. 32 is a flowchart illustrating an exemplary radiation method according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by other expressions if they achieve the same purpose.

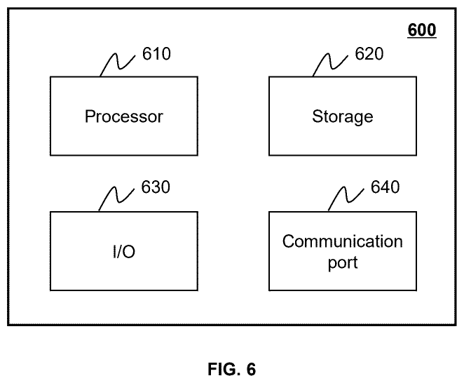

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or other storage devices. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 610 as illustrated in FIG. 6) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in a firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

For illustration purposes, the following description is provided to help better understanding. It is understood that this is not intended to limit the scope of the present disclosure. For persons having ordinary skills in the art, a certain amount of variations, changes and/or modifications may be deducted under the guidance of the present disclosure. Those variations, changes and/or modifications do not depart from the scope of the present disclosure.

In this present disclosure, the terms “radiation therapy,” “radiotherapy,” “radiation treatment,” “treatment,” and “treatment session” may be used interchangeably to refer to a therapy for treating, e.g., cancers and other ailments in biological (e.g., human and animal) tissue using radiation. The terms “treatment plan,” “therapy plan,” and “radiotherapy plan” may be used interchangeably to refer to a plan used to perform radiotherapy.

An aspect of the present disclosure relates to a radiation system. The radiation system may include a gantry, a treatment head, a detector, and a plurality of imaging sources. The treatment head, the detector, and the plurality of imaging sources may be mounted on the gantry. The treatment head may be configured to deliver a treatment beam toward an object. The plurality of imaging sources may be configured to deliver a plurality of imaging beams toward the object. At least two of the plurality of imaging sources may share the detector. The detector (also referred to as an imaging beam detector) may be configured to detect at least two of the plurality of imaging beams. The detected at least two imaging beams may be emitted by different imaging sources of the at least two imaging sources.

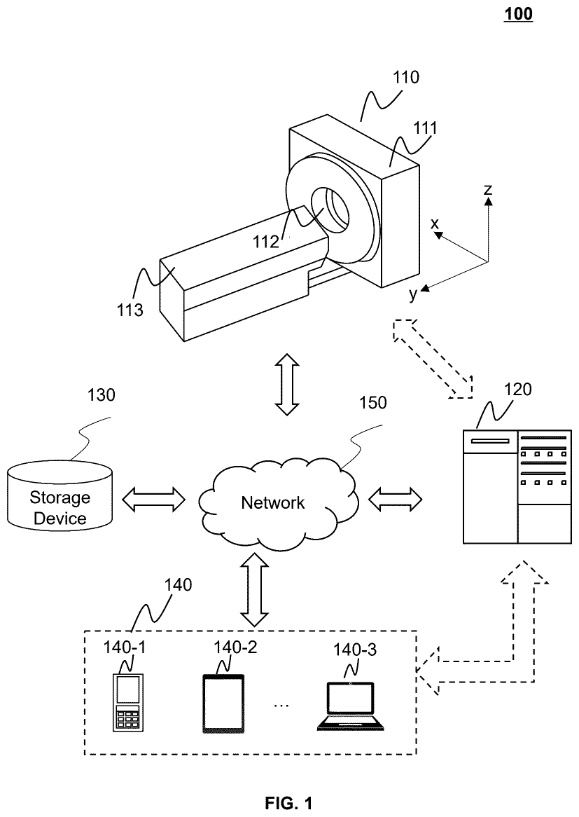

According to some embodiments of the present disclosure, the at least two of the plurality of imaging sources may share the imaging beam detector such that there may be enough space to arrange the treatment head, the plurality of imaging sources, and the imaging beam detector in a same plane, thereby bringing about one or more the following benefits. The length (e.g., along the y-direction illustrated in FIG. 1) of the gantry may be reduced, thereby making it more convenient to transport and/or set up the radiation system, and/or reducing the space needed for housing the radiation system. Besides, since the treatment head and the plurality of imaging sources are located in the same plane, the object, or a portion thereof, may be imaged during a radiotherapy at a position where the object is treated, thereby obviating the need to move the object between different treatment and imaging positions and obviating the need to perform position adjustments with respect to a treatment plan of the object, which in turn may save time and improve the utilization efficiency of the radiation system, alleviate the problems of different table sagging of the patient support between different treatment and imaging positions and resultant errors in a treatment performed at a treatment position based on imaging performed at an imaging position, and allow in-treatment imaging to facilitate in-treatment monitoring or tracking of the object, or a portion thereof, and a timely adjustment of the treatment execution accordingly.

Another aspect of the present disclosure may relate to a radiation system. The radiation system may include a plurality of imaging sources and a detector (also referred to as an imaging beam detector, e.g., a curvilinear detector). At least two of the plurality of imaging sources may share the detector. The detector may be configured to detect at least two imaging beams emitted by the at least two imaging sources, respectively. The detected at least two imaging beams may be emitted by different imaging sources of the at least two imaging sources. The radiation system may generate a pre-treatment image (e.g., a 3D image) by causing the CT imaging source of the radiation system to emit a pre-treatment imaging beam toward an object. The radiation system may determine position information (e.g., a position, a contour) of a target region of the object based on the pre-treatment image and cause the target region of the object to be positioned in the radiation system according to the position information. In some embodiments, the radiation system may also use the pre-treatment image to determine a treatment plan of the target region of the object or determine whether to adjust a treatment plan determined based on a plan image of the object.

According to another aspect of the present disclosure, the radiation system may cause, based on a treatment plan of the object, a treatment head of the radiation system to deliver at least one treatment beam toward a target region of an object to perform a radiotherapy on the target region. The radiation system may also generate at least one treatment image based on at least a portion of the at least one treatment beam detected by a detector (also referred to as a treatment beam detector, e.g., an electronic portal imaging device (EPID)) of the radiation system. Before the radiotherapy of the target region, the radiation system may generate a pre-treatment image (e.g., a 3D image) by causing the CT imaging source of the radiation system to emit a pre-treatment imaging beam toward the object. Further, the radiation system may determine, based on the pre-treatment image and the at least one treatment image, whether a delivery of the treatment beam conforms to a planned treatment beam delivery according to the treatment plan. In some embodiments, the radiation system may estimate an actual radiation dose distribution (e.g., a 2D radiation dose distribution, a 3D radiation dose distribution) of the treatment beam in the object based on the at least one treatment image. The radiation system may determine a difference between the actual radiation dose distribution and a planned radiation dose distribution in the object and then determine, based on the difference, whether the treatment beam delivery conforms to the planned treatment beam delivery according to the treatment plan. In such cases, the radiation system may achieve an in-treatment monitoring by monitoring the actual radiation dose distribution in the object in (substantially) real-time during a treatment session. If it is determined that the actual radiation dose distribution deviates from the planned radiation dose distribution, the radiation system may adjust a delivery of the treatment beam or position information (e.g., a position thereof) of the target region accordingly, thereby improving the accuracy of the radiotherapy.

According to a further aspect of the present disclosure, the radiation system may cause a treatment head of the radiation system to deliver a treatment beam toward a target region of an object based on a treatment plan of the object to perform a radiotherapy on the target region. During the radiotherapy, the radiation system may generate a plurality of groups of images (e.g., a plurality of groups of 2D images) of the object, each group at a time point. A group of images may be obtained by causing the plurality of imaging sources of the radiation system to emit a plurality of imaging beams toward the object and the detector to provide views of the object at a time point from different directions/view angles. The radiation system may track, based on the plurality of groups of images, position information (e.g., a position thereof) of the object at different time points. If it is detected that a change of the position information of the target region exceeds a threshold, the radiation system may adjust a delivery of the treatment beam or position information (e.g., a position thereof) of the target region accordingly, thereby improving the accuracy of the radiotherapy.

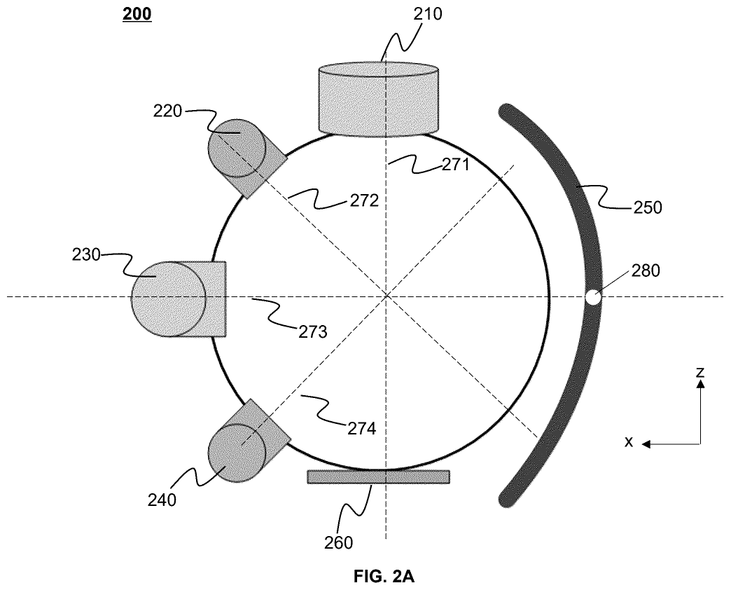

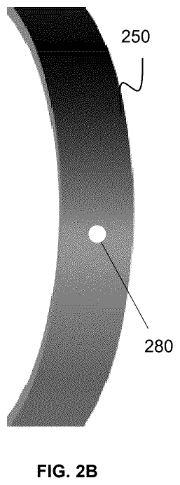

Compared to images from one (e.g., from the view perpendicular to the direction of the motion of interest) or two views (e.g., two views one of which is perpendicular to the direction of the motion of interest), the plurality of images from different views of the object may provide anatomical and/or motion information of the target region of the object with improved quality for the monitoring, thereby improving the monitoring accuracy. For example, the plurality of imaging sources may include two DR imaging sources whose axes (e.g., axes 272 and 274 illustrated in FIG. 2A) are at an angle to each other (e.g., perpendicular to each other) and a CT imaging source, thereby providing three images from three different views of the object.

There is an increasing demand for multi-modality imaging technologies in fields such as radiation delivery (e.g., radiation therapy, radiation processing, radiation verification, etc.), medical diagnostics, industrial inspection, etc. For example, in the field of radiotherapy technology, radiation therapy (radiotherapy) is a treatment in which a radiation beam (i.e., a radiation beam delivered by a radiotherapy system) passes through a portion of an object (e.g., the patient) to reach an injury or tumor (i.e., a target region) in the body of the patient. Radiotherapy plays an increasingly important role in tumor treatment. In order to provide more precise irradiation of a tumor site and better protect critical organs around the tumor, various imaging means or modalities may be applied in IGRT, such as Computed Radiography (CR) imaging, Digital Radiography (DR) imaging, Cone Beam Computed Tomography (CBCT) imaging, Fan Beam Computed Tomography (FBCT) imaging, etc. Different imaging modalities have different imaging advantages and application scenarios. The CR and DR imaging modalities have a relatively fast imaging speed, a relatively small radiation quantity, a relatively high spatial resolution, and a relatively low noise rate. The CT imaging modality have a relatively fast scanning speed, a relatively high image resolution, and can provide very fine lesion or tissue morphology. To enable a system to have the advantages of different imaging modalities, a system integrating the CT imaging and DR imaging has been proposed. However, when the CT imaging and DR imaging are integrated in a single system, it is prone to a lack of physical space and interferences between CT and DR components. Besides, if the CT imaging and DR imaging are needed to be integrated in a single system, the design cost may increase. Therefore, an imaging device integrating different imaging modalities is provided in the present disclosure. It should be noted that the imaging devices involved in various embodiments herein may be used separately as an imaging system for imaging (i.e., directly for performing an imaging operation on a target object without combining with any other device), or may be configured for IGRT or other processing when combined with other device(s) (e.g., various types of radiation devices).

In some embodiments, the plurality of imaging beams may include a CT imaging beam emitted by the CT imaging source, and the CT imaging beam may be of a fan angle achieved by adjusting an aperture of a collimator of the CT imaging source. By adjusting the fan angle of the CT imaging source, the plurality of images may be 2D images generated without performing an image reconstruction process, reducing the time for image acquisition and processing to a relatively short time (e.g., 1 millisecond, 5 milliseconds, 10 milliseconds, 50 milliseconds, 100 milliseconds), thereby improving the tracking efficiency and/or allowing an in-treatment tracking/monitoring.

In some embodiments, a tomosynthesis (also referred to as digital tomosynthesis (DTS)) imaging may be performed by emitting the plurality of imaging beams by the plurality of imaging sources. Each of the plurality of imaging sources may only need to rotate within a relatively small angle range to perform the tomosynthesis imaging; that is, the imaging source only needs to rotate for a relatively short time period, thereby improving a temporal resolution of the tracking. In some embodiments, if each of the plurality of imaging sources rotates within a same angle range as that when only one imaging source performs the tomosynthesis imaging, a quality of an image generated by tomosynthesis imaging performed by the plurality of imaging sources may be improved compared to that of an image generated by tomosynthesis imaging performed by one imaging source.

According to another aspect of the present disclosure, the radiation system may cause the plurality of imaging sources of the radiation system to emit a plurality of imaging beams at least two of which are of different energy levels. The plurality of imaging beams may include a CT imaging beam emitted by the CT imaging source, and the CT imaging beam may be of a fan angle achieved by adjusting an aperture of a collimator of the CT imaging source. The radiation system may generate an image (e.g., a multi-energy image) of the object based on at least a part of the plurality of imaging beams of different energy levels detected by the detector. The radiation system may also generate a second image (e.g., a 3D image) by causing the CT imaging source to emit a second CT imaging beam of a second fan angle achieved by adjusting the aperture of the collimator of the CT imaging source. In some embodiments, the radiation system may generate a fused image by fusing the image and the second image. The fused image may have an improved contrast of tissues (e.g., soft tissues) in and/or surrounding the target region. The radiation system may determine information of the target region (e.g., a contour of the target region, a contour of a tissue in and/or surrounding the target region) in the fused image. In some embodiments, the radiation system may also adjust a treatment plan based on the information of the target region in the fused image. Further, a treatment beam may be accurately delivered to the target region based on the determined information of the target region and the treatment plan or the adjusted treatment plan during a treatment session of the target region, thereby reducing damages to an organ or tissue in the vicinity of the target region due to exposure to treatment radiation, and/or improving the efficacy of the radiotherapy.

In some embodiments, the second fan angle may be larger than the fan angle. By dynamically adjusting a fan angle of the CT imaging source, the radiation system may generate a 3D image with more anatomical information or a 2D image within a relatively short time, thereby improving the utilization efficiency of the radiation system with a minimal or acceptable compromise of the quality of the acquired images with respect to their intended use.

FIG. 1 is a schematic diagram illustrating an exemplary radiation system according to some embodiments of the present disclosure. In some embodiments, the radiation system 100 may be configured to provide radiation therapy (e.g., stereotactic radiosurgery and/or precision radiotherapy) for lesions, tumors, and conditions anywhere in a patient where radiation treatment is indicated. In some embodiments, the radiation system 100 may include a treatment plan system (TPS), an image-guided radiotherapy (IGRT) system, etc.

As illustrated in FIG. 1, the radiation system 100 may include a radiation device 110, a processing device 120, a storage device 130, one or more terminals 140, and a network 150. The components in the radiation system 100 may be connected in one or more of various ways. Merely by way of example, the radiation device 110 may be connected to the processing device 120 through the network 150. As another example, the radiation device 110 may be connected to the processing device 120 directly as indicated by the bi-directional arrow in dotted lines linking the radiation device 110 and the processing device 120. As a further example, the storage device 130 may be connected to the processing device 120 directly or through the network 150. As still a further example, the terminal 140 may be connected to the processing device 120 directly (as indicated by the bi-directional arrow in dotted lines linking the terminal 140 and the processing device 120) or through the network 150.

In some embodiments, the radiation system 100 may perform image-guided radiation therapy (IGRT) that monitors, using X-ray imaging, a target volume (also referred to as a target region, e.g., a tumor, a lesion, etc.) to be treated inside an object (e.g., a patient). In this case, the radiation device 110 may include a treatment assembly (also referred to as a treatment device) and an imaging assembly (also referred to as an imaging device). The treatment assembly may be configured to deliver a treatment beam to the target volume to perform a radiotherapy on the target volume. The imaging assembly may be configured to perform imaging (e.g., two-dimensional (2D) imaging, three-dimensional (3D) imaging, or four-dimensional (4D) imaging) on the target volume and/or normal tissue surrounding the target volume (also referred to as “organ at risk”) before, after, or while the radiotherapy is performed. In this way, the anatomy, as well as the motion or deformation, of the target volume can be detected, and the patient's position and/or the treatment beam can be adjusted for more precise radiation dose delivery to the target volume.

In some embodiments, the imaging assembly may include a plurality of imaging sources and a detector (also referred to as an imaging beam detector, e.g., a curvilinear detector). “Plurality” used herein may refer to two or more. The plurality of imaging sources may be configured to deliver a plurality of imaging beams toward the object. In some embodiments, at least two of the plurality of imaging sources may share the detector. The detector may be configured to detect at least two imaging beams emitted by the at least two of the plurality of imaging sources. The detected at least two imaging beams may be emitted by different imaging sources of the at least two imaging sources. A detection range of the detector may encompass an aggregate field of view (FOV) of the at least two of the plurality of imaging sources. In some embodiments, the at least two imaging sources may be all the plurality of imaging sources, and the detection range of the detector may encompass an aggregate FOV of the plurality of imaging sources. In some embodiments, the at least two imaging sources may be a portion of the plurality of imaging sources, the detection range of the detector may encompass an aggregate FOV of the portion of the plurality of imaging sources. In some embodiments, an imaging dataset or an image generated based on the detected at least two imaging beams may be used to monitor the object (e.g., a motion thereof). A width (e.g., along the y-direction illustrated in FIG. 1) of the detector may be large enough to ensure that the imaging dataset or the image contains enough anatomical and/or motion information of the target region, thereby achieving a desired monitoring accuracy. In some embodiments, the width of the detector may exceed a threshold (e.g., 10 centimeters). For example, the width of the detector may be 16 centimeters.

In some embodiments, the plurality of imaging sources may include at least one (e.g., one, two) computed tomography (CT) imaging source and at least one (e.g., two, three) digital radiography (DR) imaging source. For example, the plurality of imaging sources may include a CT imaging source and two DR imaging sources. In some embodiments, the detector may be configured to detect imaging beams emitted by the CT imaging source and the two DR imaging sources. The detection range of the detector may encompass an aggregate FOV of multiple imaging sources (e.g., the CT imaging source and the two DR imaging sources in the exemplary configuration described above) of the plurality of imaging sources.

In some embodiments, the detector may be configured to detect two imaging beams emitted by the two DR imaging sources, while the radiation system may include an additional detector configured to detect an imaging beam emitted by the CT imaging source, in which the additional detector may be positioned on a gantry 111 or outside the gantry 111. For example, the additional detector may be mounted on a first rotation ring of the plurality of imaging sources or a second rotation ring different from the first rotation ring. In some embodiments, the detection range of the detector may encompass an aggregate FOV of multiple imaging sources (e.g., the two DR imaging sources in the exemplary configuration described above) of the plurality of imaging sources.

In some embodiments, the detector may be configured to detect imaging beams emitted by the CT imaging source and one of the two DR imaging sources (also referred to as a first DR imaging source), while the radiation system may include an additional detector configured to detect an imaging beam from the other of the two DR imaging sources, in which the additional detector may be positioned on the gantry 111 or outside the gantry 111. For example, the additional detector may be mounted on a first rotation ring of the plurality of imaging sources or a second rotation ring different from the first rotation ring. In some embodiments, the detection range of the detector may encompass an aggregate field of view (FOV) of the multiple imaging sources (e.g., the CT imaging source and the first DR imaging source in the exemplary configuration described above) of the plurality of imaging sources.

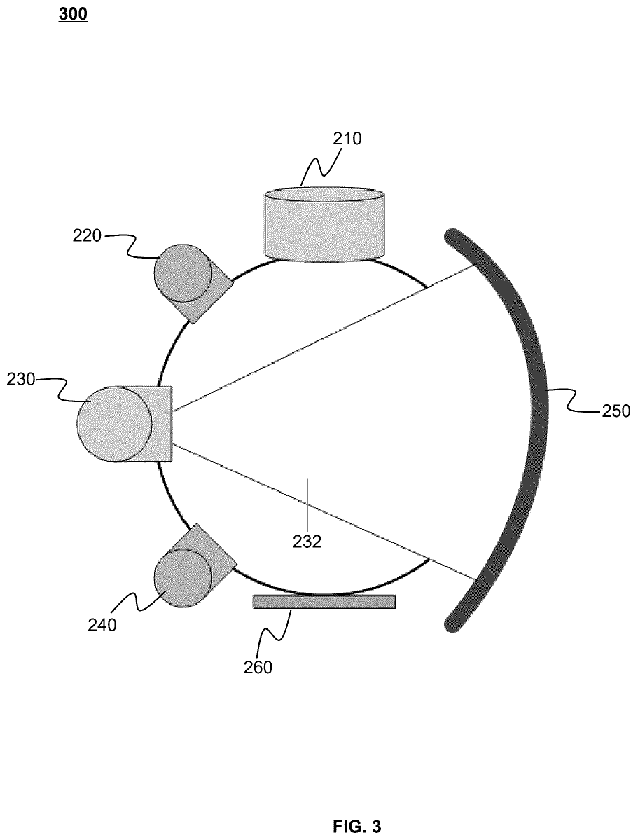

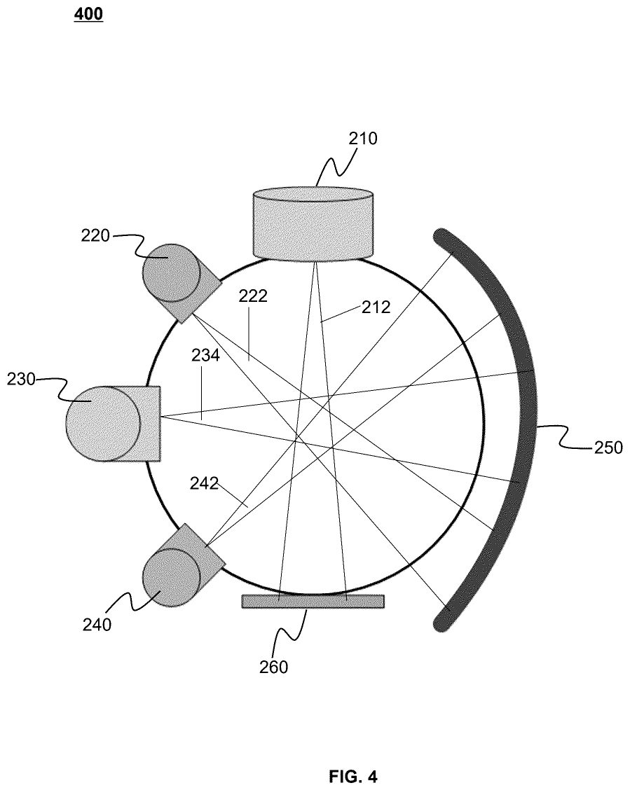

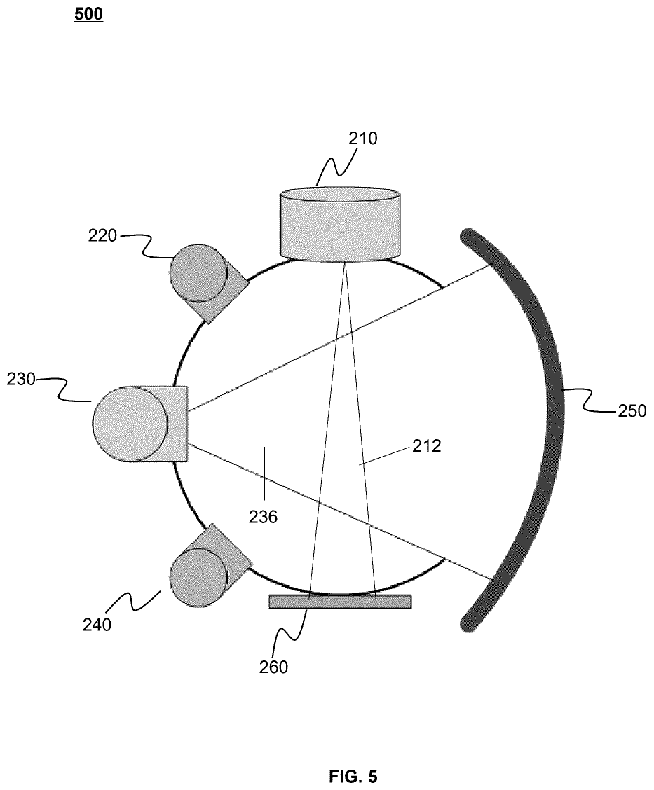

In some embodiments, the imaging beam detector may include an anti-scatter grid configured to filter out a scattered portion of the plurality of imaging beams. The anti-scatter grid may be located between the object and the imaging beam detector. In some embodiments, an orientation (e.g., an angle thereof, a position thereof) of the anti-scatter grid may correspond to a direction of a non-scattering portion of the imaging beam (also referred to as CT imaging beam) emitted by the CT imaging source. The anti-scatter grid can filter out a corresponding scattered portion of the CT imaging beam without preventing a non-scattered portion of the CT imaging beam from being detected by the imaging beam detector. In some embodiments, at least a portion of the imaging beam detector may also be configured to detect an imaging beam (or referred to as a non-CT imaging beam) emitted by a non-CT imaging source (e.g., a DR imaging source) of the plurality of imaging sources other than the CT imaging source. Since a direction of such a non-CT imaging beam may be different from the direction of the CT imaging beam (e.g., as shown in FIG. 2A, FIG. 3, FIG. 4 or FIG. 5), the anti-scatter grid configured according to the direction of the CT imaging beam may fail to filter out a scattered portion of the non-CT imaging beam, and accordingly, at least a portion of the scattered portion of the non-CT imaging beam may be detected by the imaging beam detector. In order to solve the problems, the orientation of the anti-scatter grid may need to be adjusted.

In some embodiments, the anti-scatter grid may include a plurality of portions, an orientation of each of which may be adjustable relative to at least one of the plurality of imaging sources. An orientation of at least one of the plurality of portions of the anti-scatter grid that is configured to detect the non-CT imaging beam may be adjusted to correspond to the direction of the non-CT imaging beam. In some embodiments, the orientation of each of the plurality of portions of the anti-scatter grid may be adjusted based on a piezoelectric technology. Still take the plurality of imaging sources including the CT imaging source and the at least one DR imaging source as an example. An orientation of each of at least one portion of the plurality of portions of the anti-scatter grid may be adjustable relative to at least one of the at least one DR imaging source so that the orientation of at least one of the plurality of portions of the anti-scatter grid may be adjusted to correspond to a direction of at least one imaging beam when the at least one of the at least one DR imaging source emits the at least one corresponding imaging beam toward the object.

In some embodiments, the treatment assembly may include a treatment head 112. The treatment head 112 may be configured to deliver a treatment beam toward the object to perform a radiation treatment toward a target region inside the object and/or perform imaging on a region of interest (ROI) (e.g., including the target volume and/or organs at risk (OARs)) of the object. For example, the treatment head 112 may include at least one of an acceleration tube, a treatment source (e.g., an X-ray target), a primary collimator, a filter (e.g., a flattening filter), at least one jaw, a multi-leaf collimator (MLC), etc. In some embodiments, the treatment head 112 may include an acceleration tube of particles including, for example, photons, electrons, protons, or heavy ions, etc. In some embodiments, the treatment beam may include a relatively high energy beam (e.g., an MV beam). In some embodiments, the treatment beam may include a fan beam, a cone beam, or a tetrahedron beam.

In some embodiments, the treatment head 112, the plurality of imaging sources, and the detector (i.e., the imaging beam detector) may be mounted on the gantry 111 (e.g., an O-shaped gantry). For example, the detector may be fixedly mounted on the gantry 111. As another example, the plurality of imaging sources may be fixedly mounted on the gantry 111. In some embodiments, the treatment head 112, the plurality of imaging sources, and the detector may be located in a same plane. For example, the plane may be perpendicular to an axis (also referred to as a gantry axis) of the gantry 111 along the y-axis as illustrated in FIG. 1. The treatment head 112, the plurality of imaging sources, and the imaging beam detector may rotate with the gantry 111 or independently of the gantry 111. For example, the gantry 111 may include a rotatable roller. The treatment head, the imaging beam detector, and the plurality of imaging sources may be mounted on the rotatable roller and rotate with the rotatable roller.

In some embodiments, the plurality of imaging sources may rotate independently of the treatment head 112 or the gantry 111. For illustration purposes, at least one of the plurality of imaging sources and the imaging beam detector may be operably coupled to or mounted on a rotation ring other than the gantry 111. For example, the rotation ring may be inside the gantry 111. The at least one of the plurality of imaging sources and the imaging beam detector may rotate with the rotation ring. The rotation ring may be operably coupled to, mounted on, or separated from the gantry 111. The rotation ring may rotate with or independently of the gantry 111.

In some embodiments, the plurality of imaging sources and the imaging beam detector may be stationary or substantially stationary relative to each other. As used herein, two devices, e.g., two imaging sources, an imaging source and the imaging beam detector, being stationary relative to each other indicates that the relative positioning of the two devices stay unchanged regardless of whether at least one of the two devices moves with respect to the gantry 111 or the patient support 113.

In some embodiments, the treatment head 112 may synchronously rotate with the gantry to perform a coplanar radiotherapy on the object. During the coplanar radiotherapy, radiation beams emitted by the treatment head 112 at different time points may share a same geometric plane relative toward the object. In some embodiments, a non-coplanar radiotherapy may be performed on the object by tilting (e.g., with respect to the x-direction illustrated in FIG. 1,) the gantry or rotating the patient support 113 around the z-direction illustrated in FIG. 1. During the non-coplanar radiotherapy, radiation beams emitted by the treatment head 112 at different time points may be in different geometric planes.

In some embodiments, the plurality of imaging sources may include a CT imaging source and at least one DR imaging source. In some embodiments, an angle between an axis of an imaging beam emitted by the CT imaging source and an axis of the treatment beam emitted by the treatment head 112 may be within an angular range, for example, a range between 70 degrees and 110 degrees, a range between 80 degrees and 100 degrees, a range between 85 degrees and 95 degrees, a range between 40 degrees and 120 degrees, a range between 30 degrees and 130 degrees, etc. Merely by way of example, the angle between the axis of the imaging beam emitted by the CT imaging source and the axis of the treatment beam emitted by the treatment head 112 may be (substantially) 90 degrees, e.g., 90°±10°. As another example, the angle between the axis of the imaging beam emitted by the CT imaging source and the axis of the treatment beam emitted by the treatment head 112 may be smaller than 90 degrees.

In some embodiments, an angle between the axis of the treatment beam emitted by the treatment head 112 and an axis of the detector passing a center (e.g., a point 280 illustrated in FIGS. 2A and 2B) of the detector may be within an angular range, for example, a range between 70 degrees and 110 degrees, a range between 85 degrees and 95 degrees, a range between 50 degrees and 110 degrees, a range between 40 degrees and 120 degrees, a range between 30 degrees and 130 degrees, etc. The axis of the detector may refer to an axis linking the center of the gantry 111 in a plane where the detector is located and the center of the detector. Merely by way of example, the axis of the imaging beam emitted by the CT imaging source may be perpendicular to the detector at the center of the detector; that is, the angle between the axis of the treatment beam emitted by the treatment head 112 and the axis of the detector may be (substantially) 90 degrees, e.g., 90°±10°.

In some embodiments, the detector may include a plurality of detecting units arranged in at least one row and at least one column. The center of the detector may refer to a detecting unit at an intersection of the at least one row and the at least one column. For instance, the center of the detector may refer to a detecting unit located on an intersection of the central row and the center column of the plurality of detecting units of the detector.

In some embodiments, the at least one DR imaging source may include at least two DR imaging sources. An angle between axes of two imaging beams emitted by two of the at least two DR imaging sources may be within an angular range, for example, a range between 70 degrees and 110 degrees, a range between 80 degrees and 100 degrees, a range between 85 degrees and 95 degrees, a range between 40 degrees and 120 degrees, a range between 30 degrees and 130 degrees, etc. Merely by way of example, the at least one DR imaging source may include two DR imaging sources. An angle between axes of two imaging beams emitted by the two DR imaging sources may be (substantially) 90 degrees e.g., 90°±10°. It should be noted that a count of the at least one DR imaging source may be non-limiting, for example, one, two, three, four, five, etc.

In some embodiments, each of the plurality of imaging beams may cover an imaging region. The treatment beam may cover a treatment region. The plurality of imaging sources and the treatment head 112 may be configured such that the treatment region and the plurality of imaging regions may at least partially overlap. In some embodiments, a target region (e.g., a region to be treated) of the object may be placed in an overlapping region of the treatment region and the plurality of imaging regions.

In some embodiments, the treatment head 112 and at least one of the plurality of imaging sources may be configured to emit radiation beams alternately. For example, the at least one of the plurality of imaging sources may be configured to emit at least one imaging beam when a delivery of the treatment beam toward the object is paused. In some embodiments, the treatment head 112 and the plurality of imaging sources may be positioned to move within a same rotation ring. The at least one of the plurality of imaging sources may be able to move within a range of 360 degrees of the rotation ring for one time or repeatedly.

In some embodiments, the treatment head 112 and at least one of the plurality of imaging sources may be configured to emit radiation beams concurrently. For example, the at least one of the plurality of imaging sources may be configured to emit at least one imaging beam while the treatment head 112 is delivering the treatment beam. In some embodiments, the treatment head 112 and the plurality of imaging sources may be positioned to move within a same rotation ring. The at least one of the plurality of imaging sources may be able to move independently in a limited range less than 360 degrees of the rotation ring without interfering with the treatment beam for one time or repeatedly.

In some embodiments, the treatment assembly may include a detector (also referred to as a treatment beam detector) configured to detect the treatment beam emitted by the treatment head 112 and/or at least a portion of the imaging beam(s) emitted from the plurality of imaging sources. For example, the treatment beam detector may include an electronic portal imaging device (EPID). In some embodiments, the treatment beam detector may be stationary. In some embodiments, the treatment beam detector may move independently of the treatment head 112. In some embodiments, the treatment beam detector may be positioned diametrically opposite to the treatment head 112 and rotate with the treatment head 112. In some embodiments, the treatment beam detector may be configured to detect kV beams and also MV beams. In some embodiments, the treatment beam detector may be configured to detect kV beams only or MV beams only. More descriptions of the radiation device 110 may be found elsewhere in the present disclosure (e.g., descriptions in connection with FIGS. 2A-5).

In some embodiments of the present disclosure, the radiation system 100 may include the gantry 111, the treatment head 112, a plurality of imaging sources, and at least one detector. The treatment head 112 may be configured to deliver a treatment beam toward the object. The plurality of imaging sources may be configured to deliver a plurality of imaging beams toward the object. The at least one detector may be configured to detect a plurality of imaging beams emitted by the plurality of imaging sources. In some embodiments, the plurality of imaging sources may include a first imaging source of a first type and a second imaging source of a second type that is different from the first type. For example, the first imaging source of the first type may be a CT imaging source, and the second imaging source of the second type may be a DR imaging source.

In some embodiments, the at least one detector and the plurality of imaging sources may be mounted on the gantry. The plurality of imaging beams and the treatment beam may traverse a same plane of the object. In some embodiments, the at least one detector may include one detector. The first imaging source and the second imaging source may share the detector such that the detector may be configured to detect imaging beams emitted by the first imaging source and the second imaging source.

In some embodiments, the radiation system 100 may also include a third imaging source of a third type that is different from the first type. For example, the third imaging source of the third type may be the DR imaging source. In some embodiments, the at least one detector may include one detector. The first imaging source, the second imaging source, and the third imaging source may share the detector such that the detector may be configured to detect imaging beams emitted by the first imaging source, the second imaging source, and the third imaging source.

In the present disclosure, the x-axis, the y axis, and the z-axis shown in FIG. 1 may form an orthogonal coordinate system. The x-axis and the y axis shown in FIG. 1 may be horizontal, and the z-axis may be vertical. As illustrated, the positive x-direction along the x-axis may be from the right side to the left side of the radiation device 110 seen from the direction facing the front of the radiation device 110; the positive z-direction along the z-axis shown in FIG. 1 may be from the lower part to the upper part of the radiation device 110; the positive y-direction along the y axis shown in FIG. 1 may refer to a direction in which an object is moved out of a bore of the radiation device 110.

In some embodiments, the radiation device 110 may include the gantry 111 and a patient support 113. In some embodiments, the gantry 111 may be configured to support at least one of the treatment head 112, the plurality of imaging sources, the imaging beam detector, or the treatment beam detector. The gantry 111 may be configured to rotate around an object (e.g., a patient, or a portion thereof) that is moved into or located within a field of view (FOV) (e.g., a region covered by at least one radiation beam emitted from at least one of the treatment head 112 or the plurality of imaging sources) of the radiation device 110. In some embodiments, the patient support 113 may be configured to support the object. The patient support 113 may have 6 degrees of freedom, for example, three translational degrees of freedom along three coordinate directions (i.e., x-direction, y-direction, and z-direction) and three rotational degrees of freedom around the three coordinate directions. Accordingly, the patient support 113 may move the object along a direction of the 3D coordinate system. Merely by way of example, the patient support 113 may move the object into the FOV of the radiation device 110 along the y-direction in FIG. 1.

In some embodiments, the object may be biological or non-biological. Merely by way of example, the object may include a patient, a man-made object, etc. As another example, the object may include a specific portion, organ, and/or tissue of the patient. For example, the object may include head, brain, neck, body, shoulder, arm, thorax, cardiac, stomach, blood vessel, soft tissue, knee, feet, or the like, or any combination thereof. In the present disclosure, “subject” and “object” are used interchangeably.

The network 150 may facilitate exchange of information and/or data. In some embodiments, one or more components of the radiation system 100 (e.g., the radiation device 110, the processing device 120, the storage device 130, or the terminal 140) may send information and/or data to another component(s) in the radiation system 100 via the network 150. For example, the processing device 120 may obtain a user instruction from the terminal 140 via the network 150. As another example, the processing device 120 may obtain scan data (e.g., projection data) from the radiation device 110 via the network 150. In some embodiments, the network 150 may be any type of wired or wireless network, or combination thereof. The network 150 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network), a cellular network (e.g., a Long Term Evolution (LTE) network), a frame relay network, a virtual private network (“VPN”), a satellite network, a telephone network, routers, hubs, switches, server computers, and/or any combination thereof. Merely by way of example, the network 150 may include a cable network, a wireline network, an optical fiber network, a telecommunications network, an intranet, an Internet, a local area network (LAN), a wide area network (WAN), a wireless local area network (WLAN), a metropolitan area network (MAN), a wide area network (WAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired or wireless network access points such as base stations and/or internet exchange points through which one or more components of the radiation system 100 may be connected to the network 150 to exchange data and/or information.

The terminal 140 may include a mobile device 140-1, a tablet computer 140-2, a laptop computer 140-3, or the like, or any combination thereof. In some embodiments, the mobile device 140-1 may include a smart home device, a wearable device, a smart mobile device, a virtual reality device, an augmented reality device, or the like, or any combination thereof. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a bracelet, footgear, eyeglasses, a helmet, a watch, clothing, a backpack, an accessory, or the like, or any combination thereof. In some embodiments, the smart mobile device may include a smartphone, a personal digital assistant (PDA), a gaming device, a navigation device, a point of sale (POS) device, or the like, or any combination thereof. In some embodiments, the virtual reality device and/or the augmented reality device may include a virtual reality helmet, a virtual reality glass, a virtual reality patch, an augmented reality helmet, an augmented reality glass, an augmented reality patch, or the like, or any combination thereof. For example, the virtual reality device and/or the augmented reality device may include a Google Glass, an Oculus Rift, a HoloLens, a Gear VR, etc. In some embodiments, the terminal 140 may remotely operate the radiation device 110. In some embodiments, the terminal 140 may operate the radiation device 110 via a wireless connection. In some embodiments, the terminal 140 may receive information and/or instructions inputted by a user, and send the received information and/or instructions to the radiation device 110 or to the processing device 120 via the network 150. In some embodiments, the terminal 140 may receive data and/or information from the processing device 120. In some embodiments, the terminal 140 may be part of the processing device 120. In some embodiments, the terminal 140 may be omitted.

In some embodiments, the processing device 120 may process data obtained from the radiation device 110, the storage device 130, or the terminal 140. For example, the processing device 120 may obtain projection data of an object from the radiation device 110 and generate an image of the object based on the projection data. As another example, the processing device 120 may cause one or more components (e.g., a treatment head, an imaging source, a detector, a collimator, a patient support, a gantry, etc.) of the radiation device 110 to be located at a specific position. The processing device 120 may be a central processing unit (CPU), a digital signal processor (DSP), a system on a chip (SoC), a microcontroller unit (MCU), or the like, or any combination thereof.

In some embodiments, the processing device 120 may be a single server or a server group. The server group may be centralized or distributed. In some embodiments, the processing device 120 may be local or remote. For example, the processing device 120 may access information and/or data stored in the radiation device 110, the storage device 130, and/or the terminal 140 via the network 150. As another example, the processing device 120 may be directly connected to the radiation device 110, the storage device 130, and/or the terminal 140, to access stored information and/or data. In some embodiments, the processing device 120 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

The storage device 130 may store data and/or instructions. In some embodiments, the storage device 130 may store data obtained from the terminal 140 and/or the processing device 120. For example, the storage device 130 may store one or more images generated by the processing device 120. In some embodiments, the storage device 130 may store data and/or instructions that the processing device 120 may execute or use to perform exemplary methods described in the present disclosure. For example, the storage device 130 may store instructions that the processing device 120 may execute or use to generate one or more images based on projection data. In some embodiments, the storage device 130 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 150 to communicate with one or more components of the radiation system 100 (e.g., the radiation device 110, the processing device 120, the terminal 140). One or more components of the radiation system 100 may access the data or instructions stored in the storage device 130 via the network 150. In some embodiments, the storage device 130 may be directly connected to or communicate with one or more components of the radiation system 100 (e.g., the processing device 120, the terminal 140). In some embodiments, the storage device 130 may be part of the processing device 120.

FIG. 2A is a schematic diagram illustrating an exemplary configuration of a radiation device according to some embodiments of the present disclosure. FIG. 2B is a schematic diagram illustrating an exemplary imaging beam detector according to some embodiments of the present disclosure.

According to the configuration 200 shown in FIG. 2A, the radiation device 110 may include a treatment head 210, a first DR imaging source 220, a CT imaging source 230, a second DR imaging source 240, a first detector 250 (also referred to as an imaging beam detector), and a second detector 260 (also referred to as a treatment beam detector). The CT imaging source 230 may be located between the first DR imaging source 220 and the second DR imaging source 240. The first DR imaging source 220, the CT imaging source 230, and the second DR imaging source 240 may share the first detector 250. In some embodiments, a detection range of the first detector 250 (e.g., a curvilinear detector illustrated in FIGS. 2A and 2B) may encompass an aggregate FOV of the first DR imaging source 220, the CT imaging source 230, and the second DR imaging source 240. The first detector 250 may be configured to detect at least one imaging beam (e.g., a KV beam) emitted by at least one of the first DR imaging source 220, the CT imaging source 230, or the second DR imaging source 240. For example, the first detector 250 may detect imaging beams emitted by the first DR imaging source 220, the CT imaging source 230, and the second DR imaging source 240. The second detector 260 (e.g., an EPID) may be configured to detect a treatment beam emitted by the treatment head 210. In some embodiments, the second detector 260 may be configured to detect kV beams and also MV beams. In some embodiments, the second detector 260 may be configured to detect kV beams only or MV beams only.

In some embodiments, the radiation device 200 may be configured such that a region the at least one imaging beam traverses may partially overlap a region the treatment beam traverses in an overlapping region. An object (e.g., a patient) may be positioned such that a target region (e.g., a region to be imaged or treated) of the object is located within the overlapping region.

As shown in FIG. 2A, the treatment head 210, the first DR imaging source 220, the CT imaging source 230, the second DR imaging source 240, the first detector 250, and the second detector 260 may be located in a same plane. For example, the plane may be perpendicular to a gantry axis (e.g., a center axis thereof along the y-direction) of a gantry of the radiation device 110. An angle between axes 272 and 274 of two imaging beams emitted by the first DR imaging source 220 and the second DR imaging source 240 may be 90 degrees. An angle between an axis 273 of an imaging beam emitted by the CT imaging source 230 and an axis 271 of the treatment beam emitted by the treatment head 210 may be 90 degrees. The axis 273 may be perpendicular to the first detector 250 at a center (e.g., the point 280 illustrated in FIGS. 2A and 2B) of the first detector 250.

FIG. 3 is a schematic diagram illustrating an exemplary configuration of a radiation device according to some embodiments of the present disclosure. As shown in the configuration 300, the radiation device 110 may include the treatment head 210, the first DR imaging source 220, the CT imaging source 230, the second DR imaging source 240, the first detector 250, and the second detector 260 illustrated in FIG. 2A.

According to the configuration 300, the CT imaging source 230 may be controlled to emit an imaging beam 232 toward an object. In some embodiments, the imaging beam 232 may have a relatively large fan angle. For example, the radiation range of the imaging beam 232 may be a maximum fan angle of an imaging beam emitted by the CT imaging source 230. As used herein, a fan angle of a radiation beam (e.g., the imaging beam 232) refers to an angular spread of the radiation beam emitted by a source and in a predetermined plane (e.g., a rotation plane) of the source when the source is stationary.

In some embodiments, the CT imaging source 230 may rotate or oscillate (rotating by an angle in opposite directions back and forth in an imaging scan) by a rotation angle with respect to the treatment head 210 while the imaging beam 232 is being emitted. The radiation range of the CT imaging source 230 may be a sum of the fan angle and the rotation angle. An imaging dataset corresponding to the imaging beam 232 of the radiation range (e.g., the sum of the fan angle and the rotation angle) can be used to generate a 3D image.

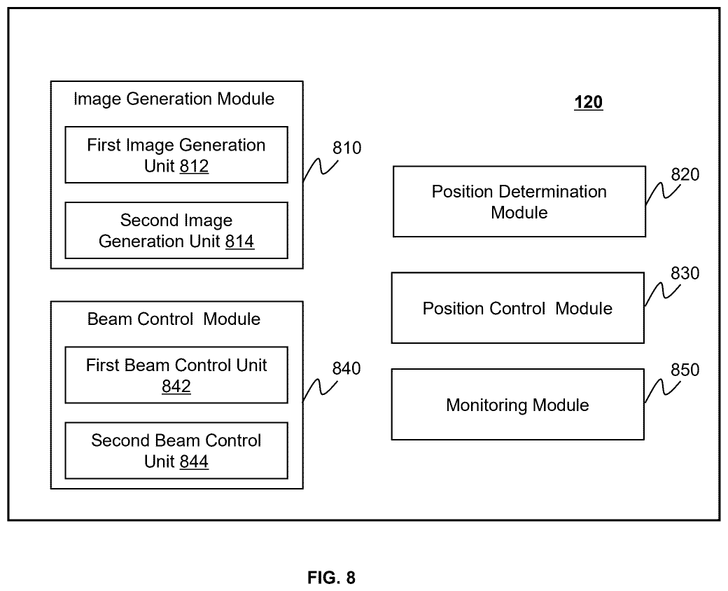

In some embodiments, an imaging dataset may be generated based on at least a portion of the imaging beam 232 detected by the first detector 250. Further, an image (e.g., a 3D image) may be generated based on at least a portion of the imaging dataset. In some embodiments, the image may be used to determine a treatment plan of a radiotherapy of the object or adjust a treatment plan determined based on a plan image of the object and/or monitor a radiotherapy of the object. More descriptions of determining the treatment plan, adjusting the treatment plan, and/or monitoring the radiotherapy based on the image may be found elsewhere in the present disclosure, for example, FIGS. 8-12 and the descriptions thereof.

FIG. 4 is a schematic diagram illustrating an exemplary configuration of a radiation device according to some embodiments of the present disclosure. As shown in the configuration 400, the radiation device 110 may include the treatment head 210, the first DR imaging source 220, the CT imaging source 230, the second DR imaging source 240, the first detector 250, and the second detector 260 illustrated in FIG. 2A or FIG. 3.

According to the configuration 400, the treatment head 210 may be controlled to emit a treatment beam 212 toward a target region (e.g., a region to be treated) of an object. The treatment beam may be delivered toward the target region to perform a radiotherapy on the target region. The first DR imaging source 220, the CT imaging source 230, and the second DR imaging source 240 may be controlled to emit imaging beams 222, 234, and 242 toward the object, respectively. As used herein, a first fan angle of the imaging beam 234 may be smaller than or equal to a second fan angle of the imaging beam 232, in which the imaging beam 232 is delivered by the CT imaging source 230 when the first DR imaging source 220 and the second DR imaging source 240 are not emitting imaging beams, while the imaging beam 234 is delivered by the CT imaging source 230 when at least one of the first DR imaging source 220 or the second DR imaging source 240 are emitting imaging beams.

In some embodiments, the radiation device 110 may include a collimator configured to adjust a fan angle of an imaging beam emitted by a corresponding imaging source (e.g., the CT imaging source). The collimator may be positioned on an imaging beam pathway of the imaging beam. The imaging beam 232 or the imaging beam 234 may be generated by adjusting an aperture of the collimator, through which a portion of the imaging beam may be delivered toward the object and/or a portion of the imaging beam may be blocked by the collimator.

In some embodiments, a region the imaging beam 222, 234, and/or 242 traverses may partially overlap a region the treatment beam 212 traverses in an overlapping region. The object may be positioned such that a target region of the object is located within the overlapping region.

In some embodiments, the delivery of one or more imaging beams (e.g., the imaging beams 222, 234, and 242) may be concurrent with the delivery of the treatment beam 212. In some embodiments, the delivery of one or more imaging beams (e.g., the imaging beams 222, 234, and 242) and the delivery of the treatment beam 212 may alternate. That is, the imaging beams 222, 234, and 242) may be delivered when the treatment beam 212 is paused. In some embodiments, an imaging dataset may be generated based on each of the imaging beams 222, 234, and 242 detected by the first detector 250. In some embodiments, an image (e.g., a 2D image) may be generated for each of the imaging datasets. For example, a first image may be generated based on at least a portion of the imaging beam 234 detected by the first detector 250. A second image may be generated based on at least a portion of the imaging beam 222 detected by the first detector 250. A third image may be generated based on at least a portion of the imaging beam 242 detected by the first detector 250.

In some embodiments, each of the imaging beams 222, 234, and 242 may impinge on a detection region of the first detector 250. In some embodiments, the plurality of detection regions may be separate from each other. The sources of signals corresponding to the imaging beams 222, 234, and 242 that are detected by the first detector 250 may be distinguishable from each other based on where the signals are detected in the plurality of separated detection regions of the first detector 250. As used herein, the source of a signal corresponding an imaging beam detected by a detector refers to the imaging source that emits the imaging beam detected by the detector (e.g., the first detector 250) and results in the signal.

In some embodiments, at least two of the plurality of detection regions may at least partially overlap. In some embodiments, the imaging beams 222, 234, and 242 may be emitted at different time points such that the sources of signals corresponding to the imaging beams 222, 234, and 242 may be distinguishable from each other. In some embodiments, the sources of signals corresponding to the imaging beams 222, 234, and 242 that are detected by the first detector 250 may be distinguishable from each other using an anti-scatter grid located between the object and the first detector 250. In some embodiments, a detection region corresponding to the imaging beam 222 and a detection region corresponding to the imaging beam 234 may have an overlapping region. The sources of signals corresponding to the imaging beams 222 and 234 may be distinguishable by adjusting an orientation of at least a portion of the anti-scatter grid. For example, the orientation of the at least a portion of the anti-scatter grid may be adjusted to filter out a portion of the imaging beam 234 impinged on the overlapping region without preventing a portion of the imaging beam 222 from being detected by the first detector 250 to determine the source of signals corresponding to the imaging beam 222. As another example, the orientation of the at least a portion of the anti-scatter grid may be adjusted to filter out a portion of the imaging beam 222 impinged on the overlapping region without preventing a portion of the imaging beam 234 from being detected by the first detector 250 to determine the source of signals corresponding to the imaging beam 234.

In some embodiments, the imaging beams 222, 234, and 242 may be of a same energy level. In some embodiments, the imaging beams 222, 234, and 242 may be of different energy levels. A multi-energy image may be generated based on the first image, the second image, and the third image. At least one of the first image, the second image, the third image, or the multi-energy image may be used to determine a treatment plan of a radiotherapy of the object or adjust a treatment plan determined based on a plan image of the object and/or monitor a radiotherapy of the object. More descriptions of determining the treatment plan of the radiotherapy of the object or adjusting the treatment plan determined based on the plan image of the object and/or monitor the radiotherapy of the object may be found elsewhere in the present disclosure, for example, FIGS. 8-12, and the descriptions thereof.

FIG. 5 is a schematic diagram illustrating an exemplary configuration of a radiation device according to some embodiments of the present disclosure. As shown in the configuration 500, the radiation device 110 may include the treatment head 210, the first DR imaging source 220, the CT imaging source 230, the second DR imaging source 240, the first detector 250, and the second detector 260 illustrated in FIGS. 2A-4.

According to the configuration 500, the CT imaging source 230 may be controlled to emit an imaging beam 236 toward an object. Similar to the imaging beam 232, the imaging beam 236 may have a relatively large fan angle. For example, the fan angle of the imaging beam 236 may be a maximum fan angle of an imaging beam emitted by the CT imaging source 230. In some embodiments, an imaging dataset may be generated based on at least a portion of the imaging beam 236 detected by the first detector 250. Further, a first image (e.g., a 3D image) may be generated based on the imaging dataset.

In some embodiments, the treatment head 210 may be controlled to emit a treatment beam 212 toward an object. The treatment beam may be delivered toward a target region of the object to perform a radiotherapy on the target region. A second image may be generated based on at least a portion of the treatment beam detected by the second detector 260. The region the imaging beam 236 traverses may partially overlap the region the treatment beam 212 traverses in an overlapping region. The object may be positioned such that a target region of the object is located within the overlapping region. In some embodiments, the first image and the second image may be used to monitor the execution of a treatment plan of a radiotherapy of the object, and/or a deviation of the execution from the treatment plan, or adjust a treatment plan determined based on a plan image of the object and/or monitor a radiotherapy of the object. More descriptions of determining the treatment plan of the radiotherapy of the object or adjust the treatment plan determined based on the plan image of the object and/or monitor the radiotherapy of the object may be found elsewhere in the present disclosure, for example, FIGS. 8-12 and the descriptions thereof.

FIG. 6 is a schematic diagram illustrating exemplary hardware and/or software components of a computing device on which the processing device 120 may be implemented according to some embodiments of the present disclosure. As illustrated in FIG. 6, the computing device 600 may include a processor 610, a storage 620, an input/output (I/O) 630, and a communication port 640.

The processor 610 may execute computer instructions (program code) and perform functions of the processing device 120 in accordance with techniques described herein. The computer instructions may include routines, programs, objects, components, signals, data structures, procedures, modules, and functions, which perform particular functions described herein. For example, the processor 610 may process data obtained from the radiation device 110, the storage device 130, the terminal 140, or any other component of the radiation system 100. In some embodiments, the processor 610 may include a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration purposes, only one processor is described in the computing device 600. However, it should be noted that the computing device 600 in the present disclosure may also include multiple processors, thus operations and/or method steps that are performed by one processor as described in the present disclosure may also be jointly or separately performed by the multiple processors. For example, if in the present disclosure the processor of the computing device 600 executes both step A and step B, it should be understood that step A and step B may also be performed by two different processors jointly or separately in the computing device 600 (e.g., a first processor executes step A and a second processor executes step B, or the first and second processors jointly execute steps A and B).

The storage 620 may store data/information obtained from the radiation device 110, the storage device 130, the terminal 140, or any other component of the radiation system 100. In some embodiments, the storage 620 may include a mass storage device, a removable storage device, a volatile read-and-write memory, a read-only memory (ROM), or the like, or any combination thereof. For example, the mass storage device may include a magnetic disk, an optical disk, a solid-state drive, etc. The removable storage device may include a flash drive, a floppy disk, an optical disk, a memory card, a zip disk, a magnetic tape, etc. The volatile read-and-write memory may include a random access memory (RAM). The RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. The ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage 620 may store one or more programs and/or instructions to perform exemplary methods described in the present disclosure.

The I/O 630 may input or output signals, data, or information. In some embodiments, the I/O 630 may enable a user interaction with the processing device 120. For example, the processing device 120 may display an image through the I/O 630. In some embodiments, the I/O 630 may include an input device and an output device. Exemplary input devices may include a keyboard, a mouse, a touch screen, a microphone, or the like, or a combination thereof. Exemplary output devices may include a display device, a loudspeaker, a printer, a projector, or the like, or a combination thereof. Exemplary display devices may include a liquid crystal display (LCD), a light-emitting diode (LED)-based display, a flat panel display, a curved screen, a television device, a cathode ray tube (CRT), or the like, or a combination thereof.

The communication port 640 may be connected to a network (e.g., the network 150) to facilitate data communications. The communication port 640 may establish connections between the processing device 120 and the radiation device 110, the storage device 130, or the terminal 140. The connection may be a wired connection, a wireless connection, or combination of both that enables data transmission and reception. The wired connection may include an electrical cable, an optical cable, a telephone wire, or the like, or any combination thereof. The wireless connection may include Bluetooth, Wi-Fi, WiMax, WLAN, ZigBee, mobile network (e.g., 3G, 4G, 5G, etc.), or the like, or a combination thereof. In some embodiments, the communication port 640 may be a standardized communication port, such as RS232, RS485, etc. In some embodiments, the communication port 640 may be a specially designed communication port. For example, the communication port 640 may be designed in accordance with the digital imaging and communications in medicine (DICOM) protocol.

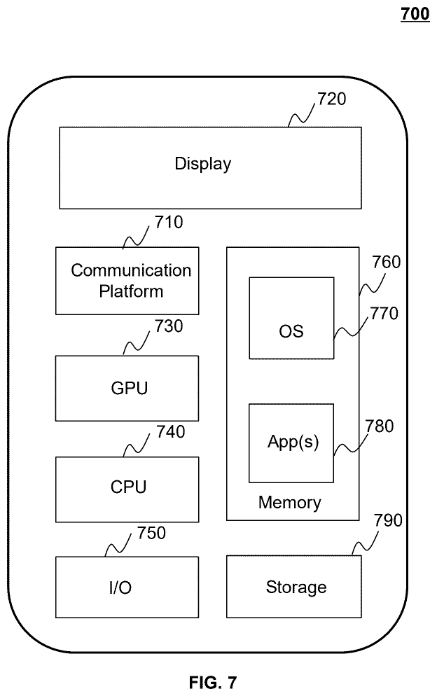

FIG. 7 is a schematic diagram illustrating exemplary hardware and/or software components of a mobile device on which the terminal 140 may be implemented according to some embodiments of the present disclosure. As illustrated in FIG. 7, the mobile device 700 may include a communication platform 710, a display 720, a graphics processing unit (GPU) 730, a central processing unit (CPU) 740, an I/O 750, a memory 760, and a storage 790. In some embodiments, any other suitable component, including a system bus or a controller (not shown), may also be included in the mobile device 700. In some embodiments, a mobile operating system 770 (e.g., IOS, Android, Windows Phone, etc.) and one or more applications 780 may be loaded into the memory 760 from the storage 790 in order to be executed by the CPU 740. The applications 780 may include a browser or any other suitable mobile apps for receiving and rendering information relating to radiation therapy or other information from the processing device 120. User interactions with the information stream may be achieved via the I/O 750 and provided to the processing device 120 and/or other components of the radiation system 100 via the network 150.

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar therewith to adapt those technologies to the radiation therapy as described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result the drawings should be self-explanatory.

FIG. 8 is a block diagram illustrating an exemplary processing device according to some embodiments of the present disclosure. The processing device 120 may include an image generation module 810, a position determination module 820, a position control module 830, a beam control module 840, and a monitoring module 850.

The image generation module 810 may be configured to generate at least one image of an object (e.g., a patient, a portion thereof). In some embodiments, the image generation module 810 may include a first image generation unit 812 and/or a second image generation unit 814.

The position determination module 820 may be configured to determine position information (e.g., a position thereof, a contour thereof) of a target region of the object in a radiation system based on at least one of the at least one image. In some embodiments, the processing device 120 may determine the position information of the target region using an image segmentation algorithm. The position control module 830 may be configured to cause the target region of the object to be positioned in the radiation system according to the position information.

The beam control module 840 may be configured to control a source (e.g., an imaging source, a treatment head of the radiation system) to emit a radiation beam (e.g., an imaging beam, a treatment beam) toward the object. In some embodiments, the beam control module 840 may include a first beam control unit 842 and/or a second beam control unit 844. The monitoring module 850 may be configured to monitor a treatment session of the object.

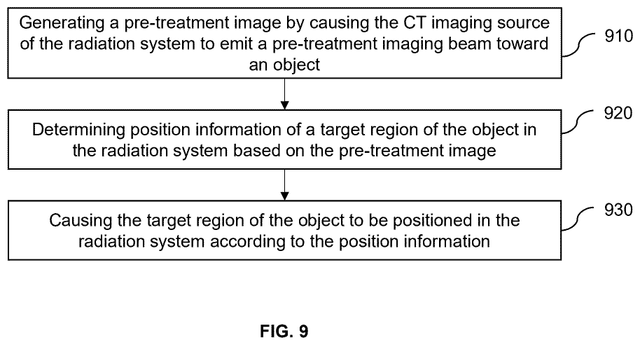

In some embodiments, the image generation module 810 (e.g., the image generation unit 812) may generate a pre-treatment image (e.g., a 3D image) by causing a CT imaging source of the radiation system to emit a pre-treatment imaging beam toward the object. The position determination module 820 may determine the position information of the target region of the object in the radiation system based on the pre-treatment image. The position control module 830 may cause the target region of the object to be positioned in the radiation system according to the position information. More descriptions regarding the functions of the modules described here may be found elsewhere in the present disclosure. See, e.g., FIG. 9 and the description thereof.

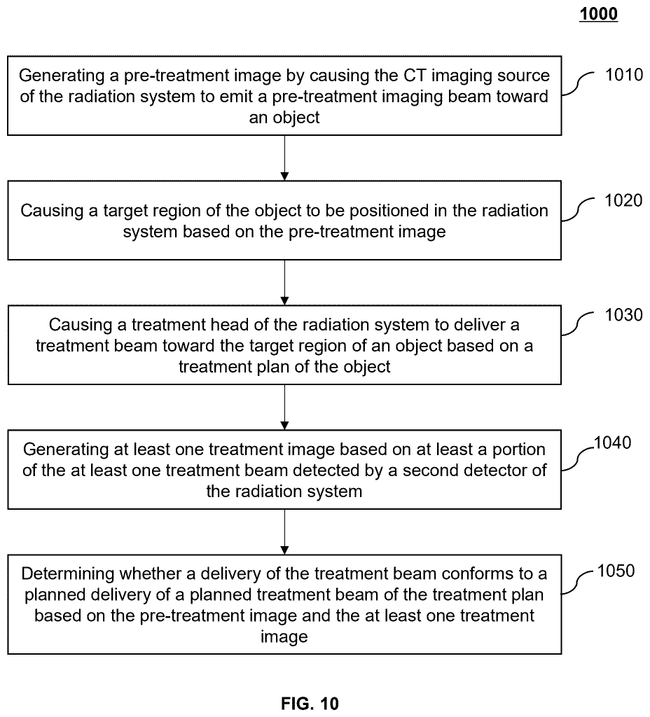

In some embodiments, the image generation module 810 (e.g., the imaging generation unit 812) may generate a pre-treatment image (e.g., a 3D image) by causing a CT imaging source of the radiation system to emit a pre-treatment imaging beam toward the object. The position control module 830 may cause the target region of the object to be positioned in the radiation system based on the pre-treatment image. The beam control module 840 (e.g., the first beam control unit 842) may cause the treatment head of the radiation system to deliver, based on a treatment plan of the object, at least one treatment beam toward the target region of the object. The image generation module 810 (e.g., the second image generation unit 814) may generate at least one treatment image based on at least a portion of the at least one treatment beam detected by a second detector of the radiation system. The monitoring module 850 may determine, based on the pre-treatment image and the at least one treatment image, whether a delivery of the treatment beam conforms to a planned treatment beam delivery according to the treatment plan. More descriptions regarding the functions of the modules described here may be found elsewhere in the present disclosure. See, e.g., FIG. 10 and the description thereof.

In some embodiments, the beam control module 840 (e.g., the first beam control unit 842) may cause the treatment head of the radiation system to deliver a treatment beam toward the target region of the object based on a treatment plan of the object. The beam control module 840 (e.g., the second beam control unit 844) may cause a plurality of imaging sources of the radiation system to emit a plurality of imaging beams toward the object and a detector (e.g., an imaging beam detector). The plurality of imaging beams may include a CT imaging beam emitted by the CT imaging source. The CT imaging beam may be of a fan angle achieved by adjusting an aperture of a collimator of the CT imaging source. The image generation module 810 (e.g., the first image generation unit 812) may generate a group of images (e.g., 2D images) of the object based on at least a part of the plurality of imaging beams detected by the detector. The position determination module 820 may determine position information of the target region based on the group of images of the object. More descriptions regarding the functions of the modules described here may be found elsewhere in the present disclosure. See, e.g., FIG. 11 and the description thereof.

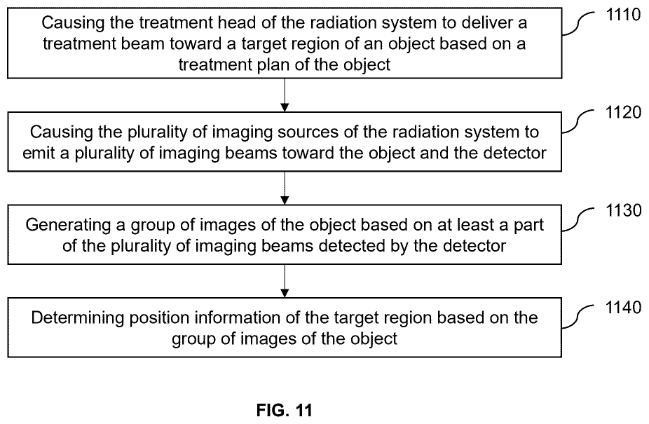

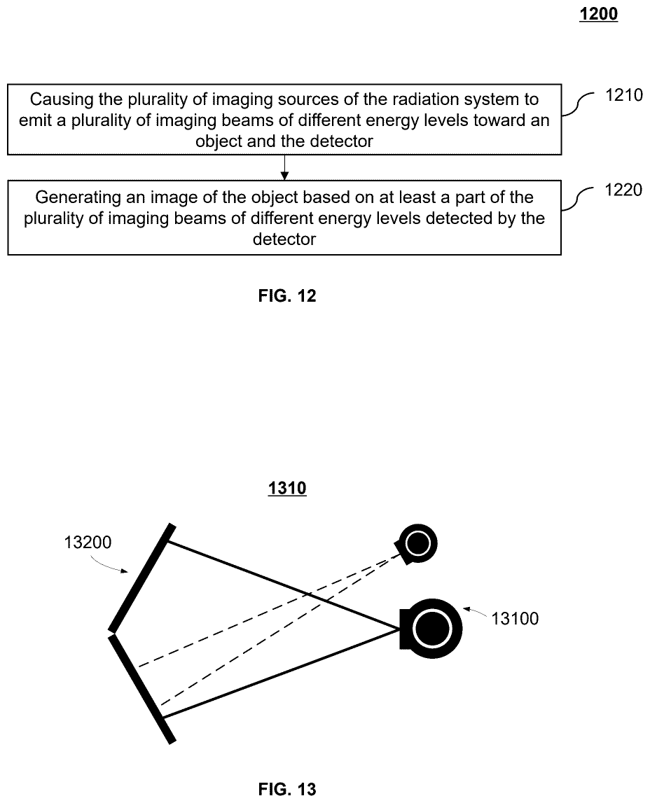

In some embodiments, the beam control module 840 (e.g., the first beam control unit 842) may cause the plurality of imaging sources of the radiation system to emit a plurality of imaging beams of different energy levels toward the object and a detector (e.g., an imaging beam detector). The plurality of imaging beams may include a CT imaging beam emitted by the CT imaging source. The CT imaging beam may be of a fan angle achieved by adjusting an aperture of a collimator of the CT imaging source. The image generation module 810 (e.g., the first image generation unit 812) may generate an image of the object based on at least a part of the plurality of imaging beams of different energy levels detected by the detector. More descriptions regarding the functions of the modules described here may be found elsewhere in the present disclosure. See, e.g., FIG. 12 and the description thereof.

In some embodiments, the processing device 120 may be unnecessary to include all the modules and/or units described above, and the processing device 120 may only include a part of the modules and/or units. For example, the processing device 120 may include the image generation module 810, the position determination module 820, and the position control module 830. As another example, the processing device 120 may include the image generation module 810, the position control module 830, the beam control module 840, and the monitoring module 850. As a further example, the processing device 120 may include the image generation module 810, the position determination module 820, and the beam control module 840. As still a further example, the processing device 120 may include the image generation module 810 and the beam control module 840.

The modules in the processing device 120 may be connected to or communicate with each other via a wired connection or a wireless connection. The wired connection may include a metal cable, an optical cable, a hybrid cable, or the like, or any combination thereof. The wireless connection may include a Local Area Network (LAN), a Wide Area Network (WAN), a Bluetooth, a ZigBee, a Near Field Communication (NFC), or the like, or any combination thereof. Two or more of the modules may be combined as a single module, and any one of the modules may be divided to two or more units.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the processing device 120 may further include a storage module. The storage module may be configured to store data generated during any process performed by any component of the processing device 120. As another example, each of the components of the processing device 120 may include a storage apparatus. Additionally, or alternatively, the components of the processing device 120 may share a common storage apparatus.

FIG. 9 is a flowchart illustrating an exemplary imaging process of a radiation system according to some embodiments of the present disclosure. The process 900 may be implemented in the radiation system 100 illustrated in FIG. 1. For example, the process 900 may be stored in the storage device 130 and/or the storage 620 in the form of instructions (e.g., an application), and invoked and/or executed by the processing device 120 (e.g., the processor 610 illustrated in FIG. 6, or one or more modules in the processing device 120 illustrated in FIG. 8). The operations of the illustrated process presented below are intended to be illustrative. In some embodiments, the process 900 may be accomplished with one or more additional operations not described, and/or without one or more of the operations discussed. Additionally, the order in which the operations of the process 900 as illustrated in FIG. 9 and described below is not intended to be limiting.

In some embodiments, the radiation system may include a plurality of imaging sources and a detector (e.g., the imaging beam detector illustrated in FIGS. 1-5, e.g., a curvilinear detector). At least two of the plurality of imaging sources may share the detector. The detector may be configured to detect at least two imaging beams emitted by the at least two different imaging sources of the plurality of imaging sources. The detected at least two imaging beams may be emitted by different imaging sources of the at least two imaging sources. The radiation system may be similar to the radiation system 100 illustrated in FIGS. 1-5, the descriptions of which are not repeated here.

In 910, the processing device 120 (e.g., the image generation module 810, e.g., the first image generation unit 812) may generate a pre-treatment image (e.g., a 3D image) by causing the CT imaging source of the radiation system to emit a pre-treatment imaging beam toward an object (e.g., a patient, or a portion thereof). In some embodiments, the processing device 120 may obtain an imaging dataset (e.g., projection data) corresponding to at least a portion of the pre-treatment imaging beam detected by the detector (e.g., the imaging beam detector illustrated in FIGS. 1-5) of the radiation system. In some embodiments, the processing device 120 may generate the pre-treatment image based on at least a portion of the imaging dataset. In some embodiments, the processing device 120 may reconstruct the pre-treatment image using a reconstruction algorithm. For example, the reconstruction algorithm may include an iterative reconstruction algorithm (e.g., a statistical reconstruction algorithm), a Fourier slice theorem algorithm, a filtered back projection (FBP) algorithm, a fan-beam reconstruction algorithm, an analytic reconstruction algorithm, or the like, or any combination thereof.

In some embodiments, the pre-treatment imaging beam may be of a fan angle achieved by adjusting an aperture of a collimator of the CT imaging source. In some embodiments, the CT imaging source may rotate or oscillate (rotating by an angle in opposite directions back and forth in an imaging scan) by a rotation angle with respect to a treatment head of the radiation system while the pre-treatment imaging beam is being emitted. The radiation range of the CT imaging source may be a sum of the fan angle and the rotation angle. An imaging dataset corresponding to the pre-treatment imaging beam of the radiation range (e.g., the sum of the fan angle and the rotation angle) can be used to generate a 3D image.

In some embodiments, a radiation device of the radiation system may have the configuration 300 in 910. As described in connection with FIG. 3, the radiation device may include the treatment head, two DR imaging sources, the CT imaging source and the imaging beam detector. The pre-treatment imaging beam may have a relatively large fan angle, in which the pre-treatment imaging beam is delivered by the CT imaging source when the two DR imaging sources are not emitting imaging beams.

In 920, the processing device 120 (e.g., the position determination module 820) may determine position information of a target region of the object in the radiation system based on the pre-treatment image. For example, the position information of the target region may include a position of the target region, a contour of the target region, etc. In some embodiments, the processing device 120 may determine the position information of the target region by segmenting the pre-treatment image using an image segmentation algorithm. For example, the image segmentation algorithm may include a thresholding algorithm, a clustering algorithm, a motion and interactive segmentation algorithm, a compression-based algorithm, a histogram-based algorithm, an edge detection algorithm, a region-growing algorithm, a model-based segmentation algorithm (e.g., a neural network model), or the like, or any combination thereof.

In 930, the processing device 120 (e.g., the position control module 830) may cause the target region of the object to be positioned in the radiation system according to the position information.

In some embodiments, the processing device 120 may generate a second pre-treatment image by causing at least one of the plurality of imaging sources to emit at least one second pre-treatment imaging beam toward the object. In some embodiments, the at least one second pre-treatment imaging beam may include at least two second pre-treatment imaging beams of a same energy level that are emitted by at least two of the plurality of imaging sources, respectively. In some embodiments, the at least one second pre-treatment imaging beam may include at least two second pre-treatment imaging beams of different energy levels that are emitted by at least two of the plurality of imaging sources, respectively.

For example, the second pre-treatment image may be a multi-energy image. In some embodiments, the at least one second pre-treatment imaging beam may include at least two second pre-treatment imaging beams that are of different energy levels. In some embodiments, the at least two second pre-treatment imaging beams of different energy levels may be emitted by at least two of the plurality of imaging sources of the radiation system. In some embodiments, the at least two second pre-treatment imaging beams of different energy levels may be emitted by one of the plurality of imaging sources that is configured to emit imaging beams of different energy levels. For example, the imaging source may emit the imaging beams of different energy levels by adjusting a voltage of the imaging source.

In some embodiments, the detector (e.g., a layer detector) may detect signals resulting from the second pre-treatment imaging beams impinging on the detector. The detector may determine the imaging sources by which the impinging imaging beams are emitted. More descriptions regarding the imaging source determination may be found elsewhere in the present disclosure. See, e.g., FIG. 4 and the description thereof.

In some embodiments, if the at least one imaging source includes the CT imaging source, the CT imaging source may be adjustably collimated by the collimator of the radiation system. A first fan angle of one of the at least one second pre-treatment imaging beam emitted by the CT imaging source may be smaller than or equal to a second fan angle of the pre-treatment imaging beam. The second pre-treatment imaging beam emitted by the CT imaging source may be of the second fan angle achieved by adjusting the aperture of the collimator of the CT imaging source.

The processing device 120 may generate the second pre-treatment image based on an imaging dataset corresponding to each of the at least two second pre-treatment imaging beams of different energy levels detected by the detector. For instance, the processing device 120 may generate at least two images (e.g., a 2D image) based on at least two imaging datasets corresponding to the at least two imaging beams and generate the second pre-treatment image by fusing the at least two images, e.g., according to a fusion algorithm. For example, the fusion algorithm may include an averaging algorithm, a Brovey algorithm, a principal component analysis (PCA) algorithm, or the like, or any combination thereof.

Further, the processing device 120 may adjust a treatment plan of the target region of the object based on the pre-treatment image and the second pre-treatment image. In some embodiments, the processing device 120 may generate a fused image by fusing the pre-treatment image and the second pre-treatment image. During the image fusion, detailed contour information of the target region and/or tissues (e.g., soft tissues) surrounding the target region may be extracted. Thus, the fused image may have an improved contrast of tissues (e.g., soft tissues) in and/or surrounding the target region.

In some embodiments, the processing device 120 may determine information of the target region in the fused image. For example, the information of the target region may include a contour of the target region in the fused image, a contour of a tissue in and/or surrounding the target region in the fused image, etc. The processing device 120 may adjust the treatment plan of the target region of the object based on the information of the target region. In some embodiments, the processing device 120 may identify a change (e.g., a position thereof, a contour thereof) of the target region based on the information of the target region in the fused image, compared to the planned information (e.g., a planned position thereof, a planned contour thereof) of the target region determined based on, e.g., a plan image of the object. In some embodiments, the plan image may be used to determine the treatment plan of the object. In response to determining that the change exceeds a threshold, the processing device 120 may adjust the treatment plan based on the information of the target region in the fused image or the change. In some embodiments, in response to determining that the change exceeds a second threshold larger than the threshold, the processing device 120 may determine a new treatment plan based on the fused image.

It should be noted the above descriptions are for illustration purposes and be non-limiting. In some embodiments, the pre-treatment image may be used as the plan image and used to determine the treatment plan of the object. In some embodiments, the pre-treatment image may be used to adjust the treatment plan regarding the target region determined based on the plan image of the object. For illustration purposes, the processing device 120 may generate a registration result by registering the pre-treatment image and the plan image and adjust the treatment plan based on the registration result. Merely by way of example, if the registration result indicates that a change (e.g., a position thereof, a contour thereof) of the target region with respect to planned information (e.g., a planned position thereof, a planned contour thereof) of the target region exceeds a threshold, the processing device 120 may adjust at least one parameter (e.g., a radiation dose, a radiation duration, a radiation dose distribution) of the target region in the treatment plan. As another example, the processing device 120 may supplement the treatment plan with at least one new parameter of a newly grown target region (e.g., a region different from (and not in) the target region, e.g., a newly grown tumor) determined based on the registration result.

In some embodiments, if the registration result indicates that the change of the target region with respect to planned information of the target region exceeds the threshold, the processing device 132 may generate a notification relating to the registration result. In some embodiments, the processing device 132 may cause the notification to be transmitted to a user (e.g., a doctor) of the radiation system and the user may provide an instruction on how to proceed further in response to the notification. In some embodiments, the processing device 132 may automatically determine how to proceed further based on the registration result.

In some embodiments, the processing device 120 may cause a treatment head of the radiation system to deliver a treatment beam toward the target region of the object in a treatment session based on the treatment plan (or an adjusted treatment plan of the object) and the position information of the target region (e.g., information of the target region in the fused image). The processing device 120 may generate a plurality of images (e.g., 2D images) of the object by causing the plurality of imaging sources of the radiation system to deliver a plurality of treatment imaging beams toward the object during the treatment session. The imaging beams may be delivered concurrently or alternately with the treatment beam. As used herein, an imaging beam delivered during a treatment session is referred to as a treatment imaging beam. A treatment imaging beam may be delivered concurrently or alternately with a treatment beam during a treatment session. As used herein, an imaging performed by an imaging source delivering an imaging beam during a treatment session is referred to as a treatment imaging. A treatment imaging may be performed to monitor the execution of the treatment plan by monitoring the position of the target region and/or tracking the delivery of the treatment beam.

In some embodiments, the position of the target region may change with time due to various motions of organs of the object, for example, cardiac motion (and its effect on other organs), respiratory motion (of the lungs and/or the diaphragm, and its effect on other organs), blood flow and motion induced by vascular pulsation, muscles contracting and relaxing, secretory activity of the pancreas, filling/emptying of bladder, rectum and digestive system, or the like, or any combination thereof. In some embodiments, the whole object may be moved along a direction (e.g., a gantry n axis of the radiation device of the treatment system). At least one of the plurality of images may be used to monitor at least one of the position and/or the motion (or movement) of the target region during the radiotherapy, a change thereof, or a rate of change thereof.

In some embodiments, the processing device 120 may determine the position of the target region based on motion information of at least one organ represented in the at least one of the plurality of images. Taking a specific organ as an example, the processing device 132 may determine motion information of the organ based on organ information of the organ represented in the at least one image. For example, the organ information may include location information of the organ, contour information of the organ, etc. In some embodiments, the processing device 132 may determine the motion information of the organ based on motion information of another organ relating to a motion of the organ. In some embodiments, at least one implant may be inserted in the vicinity of the organ and represented in at least one of the at least one image. The processing device 132 may determine the motion information of the organ based on motion information (e.g., location information, contour information) of the at least one implant.

Accordingly, the processing device 120 may adjust a delivery of the treatment beam or adjusting the position information (e.g., the position thereof) of the target region based on the at least one of the plurality of images of the object. In some embodiments, the processing device 120 may determine, based on the at least one of the plurality of images, whether any change or adjustment is needed with respect to the radiotherapy. In some embodiments, when a movement or change of the target region is detected, the processing device 120 may adjust a delivery of the treatment beam or a position of the object based on the at least one of the plurality of images of the object. For example, the processing device 120 may adjust the delivery of the treatment beam or the position of the object by adjusting at least one machine parameter of the radiation device of the radiation system. In some embodiments, the processing device 120 may adjust the position of the target region with respect to the treatment beam to allow the treatment beam to target the target region. In some embodiments, the processing device 120 may adjust a direction of the treatment beam to allow the treatment beam to target the target region. In some embodiments, the processing device 120 may adjust the treatment plan (e.g., a radiation dose of the target region, a radiation duration of the target region) and deliver an adjusted treatment beam to the object from the treatment head and based on the adjusted treatment plan. In some embodiments, the processing device 120 may cause the treatment head to pause the delivery of the treatment beam. For example, the processing device 120 may pause the delivery of the treatment beam, and then adjust the treatment head to aim at the position of the moved or changed target region. As another example, the processing device 120 may pause the delivery of the treatment beam, and then adjust the position of the target region with respect to the treatment beam to make the treatment beam target at the target region. After the delivery of the treatment beam or the position of the object is adjusted, the treatment head may resume the delivery of the treatment beam. In some embodiments, when the movement or change of the target region is detected, the treatment head may terminate the treatment beam delivery. In some embodiments, the processing device 120 may generate a notification based on the detected movement or change of the target region. In some embodiments, the notification may include information of the movement or change of the target region. The notification may be in a form of text, video, audio, or the like, or a combination thereof.

According to the systems and methods described in the present disclosure, during a radiotherapy of a target region, the processing device 120 may automatically generate and/or analyze images to record the radiotherapy, monitor the position of the target region, assess the change of the position of the target region, and/or determine how to proceed further with the radiotherapy (e.g., to continue the radiotherapy as planned, to continue the radiotherapy with a revised plan, or to terminate the radiotherapy, etc.). In some embodiments, the monitoring, assessment, and/or adjustment may be performed semi-automatically with the input of a user (e.g., a doctor). For instance, the processing device 120 may transmit the images to be presented on the terminal 140 (e.g., a display) so that the user may analyze the images and provide an instruction as to how to proceed further with the radiotherapy (e.g., to continue the radiotherapy as planned, to continue the radiotherapy with a revised plan, or to terminate the radiotherapy, etc.). As another example, the processing device 120 may first analyze the images and determine if any change occurs with respect to the target region and how much the change is. The processing device 120 may determine accordingly if any adjustment in the radiotherapy is needed. If the change of the target region or the adjustment needed in the radiotherapy is within a threshold, the processing device 120 may perform the adjustment automatically. In some embodiments, a notification may be generated when the processing device 120 makes such a determination. If the change of the target region or the adjustment needed in the radiotherapy exceeds a threshold, the processing device 120 may generate a notification to, e.g., the user to seek instructions from the user as to how to proceed further.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 10 is a flowchart illustrating an exemplary imaging process of a radiation system according to some embodiments of the present disclosure. The process 1000 may be implemented in the radiation system 100 illustrated in FIG. 1. For example, the process 1000 may be stored in the storage device 130 and/or the storage 620 in the form of instructions (e.g., an application), and invoked and/or executed by the processing device 120 (e.g., the processor 610 illustrated in FIG. 6, or one or more modules in the processing device 120 illustrated in FIG. 8). The operations of the illustrated process presented below are intended to be illustrative. In some embodiments, the process 1000 may be accomplished with one or more additional operations not described, and/or without one or more of the operations discussed. Additionally, the order in which the operations of the process 1000 as illustrated in FIG. 10 and described below is not intended to be limiting.

In some embodiments, the radiation system may include a plurality of imaging sources and a first detector (e.g., the imaging beam detector illustrated in FIGS. 1-5, e.g., a curvilinear detector). At least two of the plurality of imaging sources may share the first detector. The first detector may be configured to detect at least two imaging beams emitted by the at least two different imaging sources. The detected at least two imaging beams may be emitted by different imaging sources of the at least two imaging sources. The radiation system may be similar to the radiation system 100 illustrated in FIGS. 1-5, the descriptions of which are not repeated here.

In 1010, the processing device 120 (e.g., the image generation module 810, e.g., the first image generation unit 812) may generate a pre-treatment image (e.g., a 3D image) by causing the CT imaging source of the radiation system to emit a pre-treatment imaging beam toward an object. Operation 1010 may be similar to operation 910, the descriptions of which are not repeated here.

In 1020, the processing device 120 (e.g., the position control module 830) may cause a target region of the object to be positioned in the radiation system based on the pre-treatment image. Operation 1020 may be similar to operations 920 and 930, the descriptions of which are not repeated here.

In 1030, the processing device 120 (e.g., the beam control module 840, the first beam control unit 842) may cause a treatment head of the radiation system to deliver a treatment beam toward the target region of the object based on a treatment plan of the object. The treatment beam may be delivered toward the target region of the object to perform a radiotherapy on the target region.

In 1040, the processing device 120 (e.g., the image generation module 810, the second image generation unit 814) may generate at least one treatment image based on at least a portion of the at least one treatment beam detected by a second detector (e.g., the treatment beam detector illustrated in FIGS. 1-5) of the radiation system. In some embodiments, the processing device 120 may obtain an imaging dataset based on at least a portion of each of the at least one treatment beam detected by the second detector and further generate a treatment image (e.g., 2D image) based on the imaging dataset.

In 1050, the processing device 120 (e.g., the monitoring module 850) may determine, based on the pre-treatment image and the at least one treatment image, whether a delivery of the treatment beam (also referred to as a treatment beam delivery) conforms to a planned treatment beam delivery according to the treatment plan. In some embodiments, the at least one treatment image may include one treatment image. The processing device 120 may determine a reference treatment image based on the pre-treatment image and the treatment plan or the adjusted treatment plan of the object. For example, the treatment image and the reference treatment image may be both two-dimensional and from a same view of the object. In some embodiments, the processing device 120 may estimate a reference radiation dose distribution (e.g., a 2D radiation dose distribution) of the treatment beam in the object based on the reference treatment image and an actual radiation dose distribution (e.g., a radiation dose 2D distribution) of the treatment beam in the object based on the treatment image. Further, the processing device 120 may generate a comparison result by comparing the reference radiation dose distribution and the actual radiation dose distribution. The processing device 120 may determine whether the delivery of the treatment beam conforms to the planned treatment beam of the treatment plan based on the comparison result. In response to determining that the comparison result includes that a difference between the reference radiation dose distribution and the actual radiation dose distribution exceeds a threshold, the processing device 120 may determine that the delivery of the treatment beam fails to conform to the planned treatment beam delivery of the treatment plan. In some embodiments, the processing device 120 may further adjust a delivery of the treatment beam or position information (e.g., a position thereof) of the target region according to the process illustrated in FIG. 9.

In some embodiments, the at least one treatment image may include a plurality of treatment images from at least two different views of the object. The processing device 120 may estimate a radiation dose distribution (also referred to as an actual distribution, e.g., a 3D radiation dose distribution) of the treatment beam in the object based on the pre-treatment image and the plurality of treatment images. The processing device 120 may generate a comparison result by comparing the actual radiation dose distribution of the treatment beam and a planned radiation dose distribution in the object. The processing device 120 may determine whether the delivery of the treatment beam conforms to the planned treatment beam of the treatment plan based on the comparison result. For example, in response to determining that the comparison result includes that a difference between the actual radiation dose distribution and the planned radiation dose distribution exceeds a threshold, the processing device 120 may determine that the delivery of the treatment beam fails to conform to the planned treatment beam of the treatment plan. In some embodiments, the processing device 120 may further adjust a delivery of the treatment beam or position information (e.g., a position thereof) of the target region according to the process illustrated in FIG. 9.

In some embodiments, if the comparison result includes that the difference between the reference radiation dose distribution and the actual radiation dose distribution exceeds the threshold, the processing device 132 may generate a notification relating to the comparison result. In some embodiments, the processing device 132 may cause the notification to be transmitted to a user of the radiation system and the user may provide an instruction on how to proceed further in response to the notification. In some embodiments, the processing device 132 may automatically determine how to proceed further based on the comparison result.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 11 is a flowchart illustrating an exemplary imaging process of a radiation system according to some embodiments of the present disclosure. The process 1100 may be implemented in the radiation system 100 illustrated in FIG. 1. For example, the process 1100 may be stored in the storage device 130 and/or the storage 620 in the form of instructions (e.g., an application), and invoked and/or executed by the processing device 120 (e.g., the processor 610 illustrated in FIG. 6, or one or more modules in the processing device 120 illustrated in FIG. 8). The operations of the illustrated process presented below are intended to be illustrative. In some embodiments, the process 1100 may be accomplished with one or more additional operations not described, and/or without one or more of the operations discussed. Additionally, the order in which the operations of the process 1100 as illustrated in FIG. 11 and described below is not intended to be limiting.

In some embodiments, the radiation system may include a plurality of imaging sources, a detector (e.g., the imaging beam detector illustrated in FIGS. 1-5) (e.g., a curvilinear detector), and a treatment head. At least two of the plurality of imaging sources may share the detector. The detector may be configured to detect at least two imaging beams emitted by the at least two different imaging sources. The detected at least two imaging beams may be emitted by different imaging sources of the at least two imaging sources. In some embodiments, at least one of the plurality of imaging sources may rotate with the treatment head. In some embodiments, at least one of the plurality of imaging beams and a treatment beam of the treatment head may be emitted concurrently. The radiation system may be similar to the radiation system 100 illustrated in FIGS. 1-5, the descriptions of which are not repeated here.

In 1110, the processing device 120 (e.g., the beam control module 840, e.g., the first beam control unit 842) may cause the treatment head of the radiation system to deliver a treatment beam toward a target region of an object based on a treatment plan of the object. The treatment beam may be delivered toward the target region of the object to perform a radiotherapy on the target region.

In 1120, the processing device 120 (e.g., the image generation module 810, e.g., the first image generation unit 812) may cause the plurality of imaging sources of the radiation system to emit a plurality of imaging beams toward the object and the detector. The plurality of imaging beams may include a CT imaging beam emitted by the CT imaging source. In some embodiments, the CT imaging beam may be of a fan angle achieved by adjusting an aperture of a collimator of the CT imaging source. In some embodiments, the CT imaging source may be stationary while the CT imaging beam is emitted. A radiation range of the CT imaging source may be of the fan angle. An imaging dataset corresponding to the CT imaging beam of the fan angle can be used to generate a 2D image. In some embodiments, the CT imaging source may rotate or oscillate (rotating by an angle in opposite directions back and forth in an imaging scan) by a rotation angle with respect to a treatment head of the radiation system while the CT imaging beam is emitted. The radiation range of the CT imaging source may be a sum of the fan angle and the rotation angle. An imaging dataset corresponding to the CT imaging beam of the radiation range (e.g., the sum of the fan angle and the rotation angle) can be used to generate a 2D image.

In some embodiments, each of the plurality of imaging beams may impinge on a detection region of the detector. The plurality of detection regions may be at least partially separated from each other. In some embodiments, the detector may detect signals resulting from the imaging beams impinging on the detector. The detector may determine the imaging sources by which the impinging imaging beams are emitted. More descriptions regarding the imaging source determination may be found elsewhere in the present disclosure. See, e.g., FIG. 4 and the description thereof.

In some embodiments, a radiation device of the radiation system may have the configuration 400 in 910 and 920. As described in connection with FIG. 4, the radiation device may include the treatment head, two DR imaging sources, the CT imaging source and the imaging beam detector. The pre-treatment imaging beam may have a relatively small fan angle, in which an imaging beam is delivered by the CT imaging source when the two DR imaging sources are emitting imaging beams.

In 1130, the processing device 120 (e.g., the image generation module 810, e.g., the first image generation unit 812) may generate a group of images of the object based on at least a part of the plurality of imaging beams detected by the detector. In some embodiments, the processing device 120 may generate an imaging dataset based on at least a portion of each of the plurality of imaging beams detected by the detector and further generate an image (e.g., 2D image) based on the imaging dataset. The processing device 120 may generate the group of images based on a plurality of imaging datasets corresponding to the plurality of imaging beams. In some embodiments, at least two of the plurality of images may be from different views of the object and be two-dimensional. For instance, the different views of the object may include a sagittal view, a coronal view, a transverse view, or the like, or any combination thereof, of the object.

In 1140, the processing device 120 (e.g., the position determination module 820) may determine position information (e.g., a position thereof) of the target region based on the group of images of the object. As described in connection with FIG. 9, the position of the target region may change with time due to various motions of organs of the object. In some embodiments, the processing device 120 may determine the position information of the target region based on motion information of at least one organ represented in the groups of images. Taking a specific organ as an example, the processing device 132 may determine motion information of the organ based on organ information of the organ in the groups of images. For example, the organ information may include location information of the organ, contour information of the organ, etc. In some embodiments, the processing device 132 may determine the motion information of the organ based on motion information of another organ relating to a motion of the organ. In some embodiments, at least one implant may be inserted in the vicinity of the organ and represented in at least one of the groups of images. The processing device 132 may determine the motion information of the organ based on motion information (e.g., location information, contour information) of the at least one implant.

In some embodiments, the processing device 120 may generate a second group of images of the object by causing the plurality of imaging sources to deliver a second plurality of imaging beams toward the object and the detector. The second plurality of imaging beams may include a second CT imaging beam of the fan angle emitted by the CT imaging source. The processing device 120 may determine second position information (e.g., a position) of the target region based on the second group of images of the object. The process for generating the second group of images and determining the second position information may be similar to the process for generating the group of images and determining the position information of the target region illustrated above, the descriptions of which are not repeated here. In some embodiments, the group of images of the object may correspond to a first time point, and the second group of images of the object may correspond to a second time point that is different from the first time point.

In some embodiments, the processing device 120 may determine whether to adjust a delivery of the treatment beam and/or the position of the target region based on the position information and/or the second position information. In some embodiments, if a difference between the position information (or the second position information) of the target region determined in 1140 and initial position information of the target region exceeds a threshold, the processing device 120 may adjust the delivery of the treatment beam and/or the position of the target region based on the difference. As used herein, the initial position information of the target region may refer to position information of the target region at the beginning of delivery of the treatment beam in a same treatment session. More descriptions of adjusting the delivery of the treatment beam or the position of the target region may be found elsewhere in the present disclosure, for example, FIG. 9 and the descriptions thereof.

In some embodiments, if the difference between the position information (or the second position information) of the target region determined in 1140 and initial position information of the target region exceeds the threshold, the processing device 132 may generate a notification relating to the difference. In some embodiments, the processing device 132 may cause the notification to be transmitted to a user of the radiation system and the user may provide an instruction on how to proceed further in response to the notification. In some embodiments, the processing device 132 may automatically determine how to proceed further based on the difference.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 132 may obtain a first image based on a first imaging dataset acquired by oscillating one of the plurality of imaging sources (e.g., the CT imaging source) by a first rotation angle with respect to the treatment head of the radiation system within a first time period while a first imaging beam is emitted by the imaging source (also referred to as a tomosynthesis imaging). The processing device 132 may obtain a second image based on a second imaging dataset acquired by oscillating the imaging source by a second rotation angle with respect to the treatment head of the radiation system within a second time period while a second imaging beam is emitted by the imaging source. The first image and the second image may be used to track the position information of the object.

FIG. 12 is a flowchart illustrating an exemplary imaging process of a radiation system according to some embodiments of the present disclosure. The process 1200 may be implemented in the radiation system 100 illustrated in FIG. 1. For example, the process 1200 may be stored in the storage device 130 and/or the storage 620 in the form of instructions (e.g., an application), and invoked and/or executed by the processing device 120 (e.g., the processor 610 illustrated in FIG. 6, or one or more modules in the processing device 120 illustrated in FIG. 8). The operations of the illustrated process presented below are intended to be illustrative. In some embodiments, the process 1200 may be accomplished with one or more additional operations not described, and/or without one or more of the operations discussed. Additionally, the order in which the operations of the process 1200 as illustrated in FIG. 12 and described below is not intended to be limiting.

In some embodiments, the radiation system may include a plurality of imaging sources and a detector (e.g., the imaging beam detector illustrated in FIGS. 1-5, e.g., a curvilinear detector). At least two of the plurality of imaging sources may share the detector. The detector may be configured to detect at least two imaging beams emitted by the at least two imaging sources. The detected at least two imaging beams may be emitted by different imaging sources of the at least two imaging sources. The radiation system may be similar to the radiation system 100 illustrated in FIGS. 1-5, the descriptions of which are not repeated.

In 1210, the processing device 120 (e.g., the beam control module 840, the first beam control unit 842) may cause the plurality of imaging sources of the radiation system to emit a plurality of imaging beams of different energy levels toward an object and the detector. The plurality of imaging beams may include a CT imaging beam emitted by the CT imaging source, and the CT imaging beam is of a fan angle achieved by adjusting an aperture of a collimator of the CT imaging source. In some embodiments, the CT imaging source may be stationary while the CT imaging beam is emitted. A radiation range of the CT imaging source may be the fan angle. In some embodiments, the CT imaging source may rotate or oscillate (rotating by an angle in opposite directions back and forth in an imaging scan) by a rotation angle with respect to a treatment head of the radiation system while the CT imaging beam is emitted. The radiation range of the CT imaging source may be a sum of the fan angle and the rotation angle. An imaging dataset corresponding to the CT imaging beam of the radiation range (e.g., the sum of the fan angle and the rotation angle) can be used to generate a 2D image.

In some embodiments, each of the plurality of imaging beams may impinge on a detection region of the detector. The plurality of detection regions may be at least partially separated from each other. In some embodiments, the detector may detect signals resulting from the imaging beams impinging on the detector. The detector may determine the imaging sources by which the impinging imaging beams are emitted. More descriptions regarding the imaging source determination may be found elsewhere in the present disclosure. See, e.g., FIG. 4 and the description thereof.

In 1220, the processing device 120 (e.g., the image generation module 810, the first image generation unit 812) may generate an image (e.g., a multi-energy image) of the object based on at least a part of the plurality of imaging beams of different energy levels detected by the detector. In some embodiments, the processing device 120 may generate a primary image of the object based on an imaging dataset corresponding to each of the plurality of imaging beams detected by the detector. The processing device 120 may generate the image by fusing at least two of the plurality of primary images.

In some embodiments, the processing device 120 may cause the CT imaging source to emit a second CT imaging beam of a second fan angle achieved by adjusting the aperture of the collimator of the CT imaging source. The second fan angle may be larger than the fan angle described in 1210. For example, an imaging dataset corresponding to the second CT imaging beam of the second fan angle can be used to reconstruct a 3D image, and an imaging dataset corresponding to the CT imaging beam of the fan angle can be used to generate a 2D image. In some embodiments, the second CT imaging beam may be emitted before a radiotherapy of the target region of the subject or during the radiotherapy of the target region of the subject.

Further, the processing device 120 (e.g., the second image generation unit 814) may generate a second image based on at least a part of the second CT imaging beam detected by the detector. The processing device 120 may generate a fused image by fusing the image and the second image. Compared to the image or the second image, the fused image may have an improved contrast of tissues (e.g., soft tissues) in and/or surrounding the target region. The processing device 120 may determine information of the target region in the fused image. For example, the information of the target region may include a contour of the target region, a contour of a tissue in and/or surrounding the target region, etc. In some embodiments, the processing device 120 may adjust a treatment plan regarding the target region of the object based on the information of the target region in the fused image. More descriptions of adjusting the treatment plan may be found elsewhere in the present disclosure, for example, FIG. 9 and the descriptions thereof.

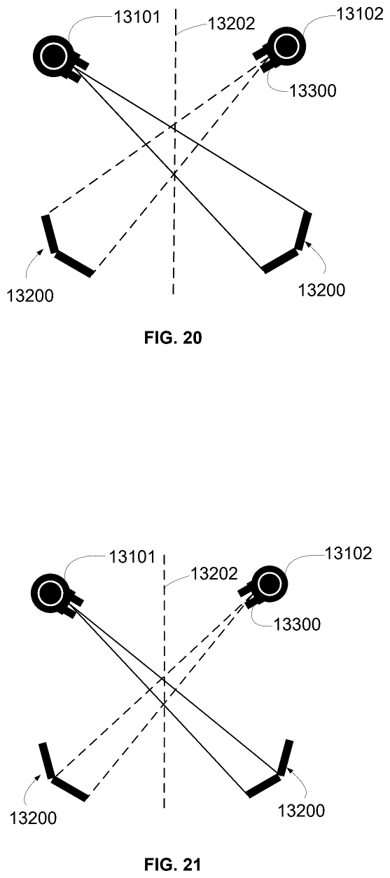

As shown in FIG. 13, the present disclosure may provide an imaging device 1310 including one or more imaging sources 13100 and one or more detectors 13200. The imaging source(s) may be configured to emit at least two different types of imaging beams. In some embodiments, the one or more detectors may include one detector. In some embodiments, the one detector may have a bent or curved shape. In some embodiments, the one or more detectors 13200 include two or more detectors. In some embodiments, the two or more detectors may be combined by joining. In some embodiments, at least one of the detectors may be set separately (as shown in FIGS. 23-25). In some embodiments, the one or more detectors may include at least two sets of detectors that are respectively disposed on two sides of a preset axis (e.g., the detectors disposed on two sides of the axis 13202 where the target object is located, as shown in FIGS. 20-25). Each set of the two sets of detectors may include one or more detectors. The detectors 13200 may be configured to receive the at least two different types of imaging beams emitted by the one or more imaging sources 13100. The detectors 13200 may generate imaging signals based on the received imaging beams. In some embodiments, the imaging device 1310 may further include one or more processing devices (e.g., the processing device 120). The processing device(s) may generate images of at least two different modalities or with at least two different energy levels based on imaging data corresponding to the imaging signals.

In some embodiments, the imaging device 1310 may include one imaging source 13100. In some embodiments, the one imaging source may be configured to be switched between at least two imaging modalities. Each of the at least two imaging modalities may correspond to emission of one type of the at least two different types of imaging beams. In some embodiments, the imaging device 1310 may include two or more imaging sources 13100. Each of the two or more imaging sources may be configured to emit a corresponding one type of the at least two different types of imaging beams. In some embodiments, each imaging source 13100 may emit one type of imaging beams. In some embodiments, each imaging source 13100 may emit at least two different types of imaging beams. That is, the at least two different types of imaging beams may be emitted from the imaging source 13100, i.e., the imaging source 13100 may have at least two different imaging modalities. In some embodiments, each of the two or more imaging sources may be configured to be switched between at least two imaging modalities. Each of the at least two imaging modalities corresponding to emission of one type of the at least two different types of imaging beams, such that the two or more imaging sources emit the at least two different types of imaging beams. Merely by way of example, the imaging source 13100 may be or include an X-ray source (e.g., a bulb tube), a y-ray source, a neutron source, etc. An imaging beam refers to a beam of rays used to perform imaging on the target object. The at least two different types of imaging beams may be imaging beams of different energies, imaging beams of different shapes, and imaging beams of different categories. Optionally, the at least two different types of imaging beams may include cone beams, fan beams, etc. Alternatively, or additionally, the at least two different types of imaging beams may include X-rays, gamma rays, and/or neutron beams, etc. As long as functions of the imaging sources 13100 are realized, a count, type, structure, etc., of the imaging source(s) 13100 included in the imaging device 1310 and the imaging beams emitted by the imaging source(s) 13100 are not limited in the present disclosure. In some embodiments, the two or more imaging sources may be configured to emit the at least two different types of imaging beams simultaneously, alternately, or asynchronously.

In some embodiments, the imaging device 1310 may further include at least one beam limiter. In some embodiments, each of the at least one beam limiter may be coupled to or mounted on an imaging source. Each of the at least one beam limiter may be configured to adjust an imaging beam emitted by an imaging source of the one or more imaging sources that corresponds to the each of the at least one beam limiter. By adjusting the beam limiter, the imaging source may emit two or more different types of imaging beams to realize different imaging modalities.

In some embodiments, the imaging device 1310 may include at least two imaging sources 13100 (i.e., in addition to the two imaging sources 13100, the imaging device 1310 may further include one or more imaging sources of the same type or different types, which is not shown in the figures). In some embodiments, the one or more imaging sources may include a computed tomography (CT) imaging source and at least one digital radiography (DR) imaging source. In some embodiments, the one or more imaging sources include two DR imaging sources. In some embodiments, one of the imaging sources 13100 may emit imaging beams of a specified type. For example, one of the imaging sources 13100 is a CT imaging source, and another one of the imaging sources 13100 is a DR imaging source.

In some embodiments, the imaging device 1310 may include one imaging source 13100 configured to emit the at least two different types of imaging beams. For example, the imaging source 13100 may emit imaging beams that may be switched between the FBCT imaging beams (fan beams) and the CBCT imaging beams (cone beams). Merely by way of example, the emission of the plurality of types of imaging beams (e.g., FBCT imaging beams, CBCT imaging beams, etc.) may be realized using one single imaging source 13100 by disposing a movable beam limiter 13300 (e.g., made of a material that shields the imaging beams, such as tungsten alloy, depleted uranium, etc., that shield X-rays) on an outlet side of the imaging beams emitted by the imaging source 13100, moving the beam limiter 13300, and/or regulating beam parameters of the imaging source 13100, etc., according to a type of the imaging beams to be emitted.

The detector 13200 may be of various types of detectors that are configured to receive imaging beams emitted by the one or more imaging sources 13100. The detector 13200 may be an amorphous selenium detector or an amorphous silicon detector. The detector 13200 may be a flat panel detector or an arc detector. The plurality of detectors 13200 may be joined and combined into one large detector. Each detector 13200 included in the imaging device 1310 may include a plurality of array units or pixels. A detection surface of each detector 13200 may be square, rectangular, curved, or in any other shape. The plurality of detectors 13200 may be joined to form a detection surface of the detector. The detection surface may face the imaging source(s) 13100. The detection surface may be rectangular, square, curved, or in any other irregular shape. In some embodiments, the plurality of detectors 13200 may be different from an array of detector units that are configured as a single piece. A count and type of the detector(s) 13200, a joining mode of the plurality of detectors 13200, and the shape of the detection surface of the joined detector are not limited in the embodiment, as long as functions thereof are realized.

Each imaging source 13100 may be disposed relative to a detector 13200 corresponding to the each imaging source 13100. The imaging beams emitted by each imaging source 13100 may be projected onto the corresponding detector 13200 through the target object. One imaging source 13100 may correspond to one of the plurality of detectors 13200, or may correspond to at least two of the plurality of detectors 13200. That is, the imaging beams emitted by one imaging source 13100 may be projected onto one detector 13200 or at least two of the plurality of detectors 13200. In the case where the imaging device 1310 includes the plurality of imaging sources 13100, the detectors 13200 corresponding to the plurality of imaging sources 13100 may be all the same, may be partially the same, or may be all different. In some embodiments, the plurality of imaging sources 13100 may share one detector 13200.

The imaging device 1310 may include the one or more imaging sources 13100 and the plurality of detectors 13200 combined by joining. The plurality of detectors 13200 may be configured to receive at least two different types of imaging beams emitted by the one or more imaging sources 13100. The imaging source 13100 included in the imaging device 1310 may emit the at least two different types of imaging beams (e.g., at least two different types (corresponding to different imaging modalities) of imaging sources may be integrated into one imaging device 1310), thereby improving the practicability of the imaging device 1310. In addition, the detectors in the imaging device 1310 configured to receive the at least two different types of imaging beams emitted by the one or more imaging sources 13100 may be obtained by joining the plurality of detectors 13200, so that the detector(s) 13200 is designed quickly, at a low cost and has relatively high flexibility.

In some embodiments, the plurality of detectors 13200 may be joined to form the detection surface, and at least a portion of the detection surface may be bent or curved. For example, in the case where the imaging device 1310 includes two detectors 13200, the two detectors 13200 may be joined to form a detection surface, and the detection surface may be bent, curved, or in other shapes (e.g., a shape formed by a combination of a flat panel detector and a curved detector). In the case where the imaging device 1310 includes more than two detectors 13200, the plurality of detectors 13200 are joined to form a detection surface, and at least a portion of the detection surface may be bent or curved (e.g., a partial region of the detection surface may be curved, and other regions may be bent or rectangular). In some embodiments, the plurality of detectors 13200 may be directly joined to form a completely bent or curved shape.

In some embodiments, the plurality of detectors 13200 in the imaging device 1310 may be joined to form the detection surface, and at least a portion of the detection surface may be bent or curved, so that the plurality of detectors 13200 may better receive the imaging beams emitted by the imaging source 13100, thereby obtaining a more accurate image. Moreover, if the at least a portion of the detection surface is bent or curved, the plurality of detectors 13200 may occupy a relatively small space, and may be easy to be integrated into the imaging device 1310. In addition, in the case where the imaging device 1310 is rotatable, the configuration of being bent or curved may reduce a centrifugal force and maintain a dynamic equilibrium easily compared to a planarly shaped detector, which facilitates the high-speed rotation of the imaging device 1310.

In some embodiments, the one or more imaging sources 13100 may include a first imaging source 13101 (or a first type of imaging source) and a second imaging source 13102 (or a second type of imaging source). In some embodiments, a first axis (e.g., a central axis) of the first type of imaging source and a second axis (e.g., a central axis) of the second type of imaging source may have a preset angle. The preset angle may be equal to or less than 90 degrees. The second imaging source 13102 may be disposed on either side of an axis (e.g., a central axis) of the first imaging source 13101, or the first imaging source 13101 and the second imaging source 13102 may be disposed on two sides of a preset axis, respectively. In some embodiments, the imaging sources 13100 may include a first imaging source 13101 (or a first type of imaging source) and at least two second imaging sources 13102 (or at least two second type of imaging sources). In some embodiments, the at least two second type of imaging sources may be respectively disposed on two sides of the first axis of the first type of imaging source.

The first imaging source 13101 and the second imaging source 13102 may be two imaging sources that each emit a specified type of imaging beams, and the specified types of imaging beams emitted by the two imaging sources may be different (e.g., the first imaging source 13101 may be a CT imaging source and the second imaging source 13102 may be a DR imaging source). In some embodiments, each of the two imaging sources can emit at least two different types of imaging beams (e.g., each of the first imaging source 13101 and the second imaging source 13102 may switchably emit CT imaging beams and DR imaging beams). In some embodiments, one of the two imaging sources may emit a specified type of imaging beams, and the other one of the two imaging sources may switchably emit at least two different types of imaging beams (e.g., the first imaging source 13101 may be a DR imaging source, and the second imaging source 13102 may switchably emit CT imaging beams and DR imaging beams), which is not limited in the embodiment as long as the functions of the first imaging source 13101 and the second imaging source 13102 are realized.

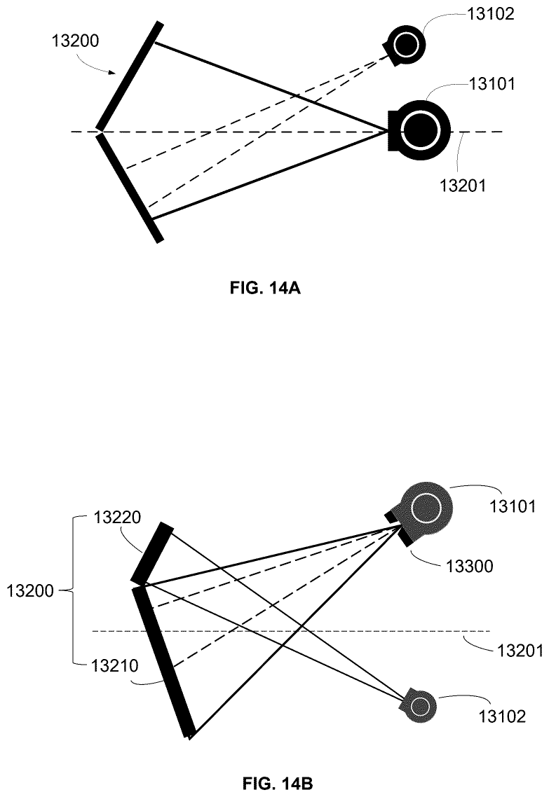

As shown in FIG. 14A, in the case where the one or more imaging sources 13100 include one first imaging source 13101 and one second imaging source 13102, the first imaging source 13101 may be disposed at any position on the axis of symmetry 13201 of the plurality of detectors 13200, the second imaging source 13102 may be disposed on either side of an axis (e.g., a central axis) of the first imaging source 13101. That is, the second imaging source 13102 may be disposed on either side of the axis of symmetry 13201 of the plurality of detectors 13200. If an axis of the first imaging source 13101 is in a horizontal direction, the second imaging source 13102 may be disposed above the axis of the first imaging source 13101 or disposed below the axis of the first imaging source 13101.

In some embodiments, the first imaging source 13101 is a CT imaging source, the second imaging source 13102 is a DR imaging source, the second imaging source 13102 may be disposed above the axis of the first imaging source 13101. The imaging beams emitted by the CT imaging source may be projected onto two detectors 13200, and the imaging beams emitted by the DR imaging source may be projected onto one detector 13200.

In the case where the one or more imaging sources 13100 include one first imaging source 13101 and one second imaging source 13102, the first imaging source 13101 and the second imaging source 13102 may be respectively disposed on two sides of a preset axis. The preset axis may be the axis of symmetry 13201 of a joined structure of the plurality of detectors 13200, or may be an axis where the target object is located. The axis where the target object is located refers to an axis in a head-to-toe direction of the target object.

As shown in FIG. 14B, the first imaging source 13101 and the second imaging source 13102 may be disposed on two sides of the axis of symmetry 13201 of a joined structure of detectors 13200. If the axis of symmetry 13201 of the joined structure of the detectors 13200 is in a horizontal direction, the first imaging source 13101 may be disposed above the preset axis and the second imaging source 13102 may be disposed below the preset axis. Alternatively, the first imaging source 13101 may be disposed below the preset axis and the second imaging source 13102 may be disposed above the preset axis. In some embodiments, the first imaging source 13101 is a CT imaging source, and the second imaging source 13102 is a DR imaging source.

In some embodiments, the imaging device 1310 may include two or more detectors 13200. The two or more detectors 13200 may include one or more first detectors 13210, and one or more second detectors 13220. The first detector 13210 and the second detector 13220 may have different areas. The first detector 13210 may have a relatively large area, and the second detector 13220 may have a relatively small area.

The first detector 13210 may receive radiation beams emitted from the first imaging source 13101 and generate corresponding imaging signals based on the received beams. In some embodiments, the imaging device 1310 may further include a beam limiter 13300 configured to cooperate with the first imaging source 13101. The beam limiter 13300 may be disposed on one side of the first imaging source 13101 facing the detectors 13200. The beam limiter 13300 may be configured to adjust the imaging beams emitted by the first imaging source 13101. The beam limiter 13300 may be configured to adjust a fan angle and/or a width of the imaging beams emitted from the first imaging source 13101 to realize various imaging modalities of the first imaging source 13101. The various imaging modalities of the first imaging source 13101 may include a DR imaging modality, a CBCT imaging modality, a flat panel CT imaging modality, etc. Through adjusting the beam limiter 13300, the first imaging source 13101 and the first detector 13210 may realize any one of the various imaging modalities and/or switch between the various imaging modalities.

Merely by way of example, through adjusting the beam limiter 13300, the first imaging source 13101 may emit large field cone beams, and the first detector 13210 with the relatively large area may realize acquisition of CBCT imaging data. In this case, the second imaging source 13102 and the second detector 13220 may not work under this mode. As another example, through adjusting the beam limiter 13300, the first imaging source 13101 may emit large field fan beams, and the first detector 13210 with the relatively large area may realize acquisition of flat panel CT imaging data. In this case, the second imaging source 13102 and the second detector 13220 may not work under this mode. As a further example, through adjusting the beam limiter 13300, the first imaging source 13101 may emit beams with a similar or same field as that of the second imaging source 13102, and the first detector 13210 with the relatively large area may realize acquisition of DR imaging data. In this case, the second imaging source 13102 and the second detector 13220 may work synchronously or may not work under this mode.

The second detector 13220 with the relatively small area may receive radiation beams emitted from the second imaging source 13102 and generate corresponding imaging signals (i.e., DR imaging data) based on the received beams.

In some embodiments, the first imaging source 13101 and the second imaging source 13102 may emit beams with a similar or same field, and a combination of the first detector 13210 with the relatively large area and the second detector 13220 with the relatively small area may realize acquisition of DR imaging data.

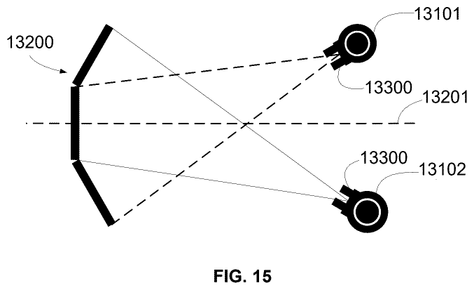

As shown in FIGS. 15-19, the first imaging source 13101 and the second imaging source 13102 may be disposed on two sides of the axis of symmetry 13201 of a joined structure of three detectors 13200. If the axis of symmetry 13201 of the joined structure of the detectors 13200 is in a horizontal direction, the first imaging source 13101 may be disposed above the preset axis and the second imaging source 13102 may be disposed below the preset axis.

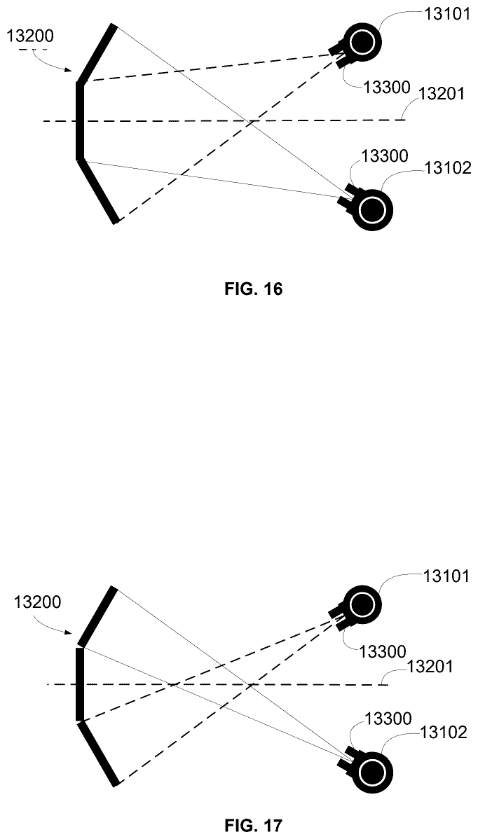

In some embodiments, the imaging device 1310 may include three detectors 13200, the first imaging source 13101 and the second imaging source 13102 may emit imaging beams corresponding to the CT imaging modality and imaging beams corresponding to the DR imaging modality. In FIGS. 15 and 16, the imaging modalities of the first imaging source 13101 and the second imaging source 13102 may be both the CT imaging modality, and the first imaging source 13101 and the second imaging source 13102 may share one detector in the middle of the three detectors 13200. For example, the imaging beams emitted by the first imaging source 13101 may be projected onto the one detector in the middle of the three detectors 13200 and one of the three detectors 13200 that is away from the first imaging source 13101. The imaging beams emitted by the second imaging source 13102 may be projected onto the one detector in the middle of the three detectors 13200 and one of the three detectors 13200 that is away from the second imaging source 13102.

In FIG. 17, the imaging modality of the first imaging source 13101 and the imaging modality of the second imaging source 13102 may be the DR imaging modality, the imaging beams emitted by the first imaging source 13101 may be projected onto one of the three detectors 13200 that is far away from the first imaging source 13101, and the imaging beams emitted by the second imaging source 13102 may be projected onto one of the three detectors 13200 that is far away from the second imaging source 13102.

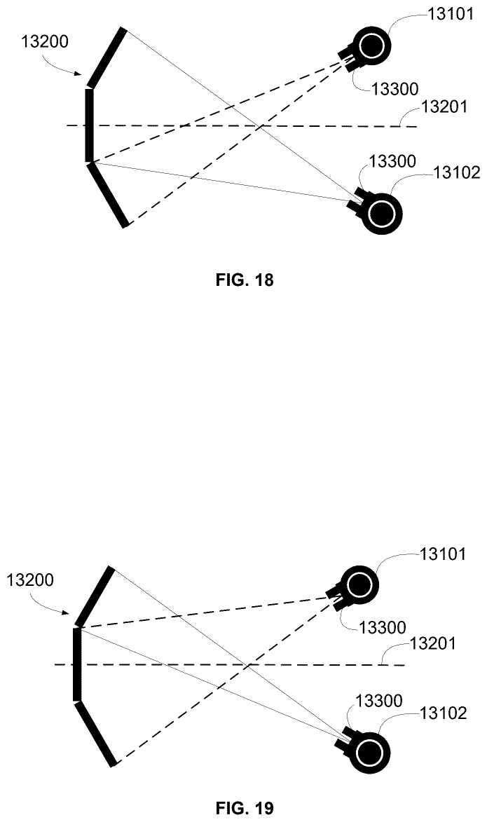

In FIG. 18, the imaging modality of the first imaging source 13101 may be the DR imaging modality, the imaging modality of the second imaging source 13102 may be the CT imaging modality, the imaging beams emitted by the first imaging source 13101 may be projected onto one of the three detectors 13200 that is far away from the first imaging source 13101, and the imaging beams emitted by the second imaging source 13102 may be projected onto one detector in the middle of the three detectors 13200 and one of the three detectors 13200 that is far away from the second imaging source 13102.

In FIG. 19, the imaging modality of the first imaging source 13101 may be the CT imaging modality, the imaging modality of the second imaging source 13102 may be the DR imaging modality, the imaging beams emitted by the first imaging source 13101 may be projected onto one detector in the middle of the three detectors 13200 and one of the three detectors 13200 that is far away from the second imaging source 13102, and the imaging beams emitted by the second imaging source 13102 may be projected onto one of the three detectors 13200 that is far away from the second imaging source 13102.

As shown in FIGS. 20-25, the first imaging source 13101 and the second imaging source 13102 may be respectively disposed on two sides of an axis 13202 where the target object is located. If the axis 13202 is in a vertical direction, the first imaging source 13101 may be disposed on the left side of the preset axis (e.g., the axis 13202) and the second imaging source 13102 may be disposed on the right side of the preset axis.

In some embodiments, the imaging device 1310 may include four detectors 13200, the first imaging source 13101 and the second imaging source 13102 may emit imaging beams corresponding to the CT imaging modality and imaging beams corresponding to the DR imaging modality. In FIG. 20, both the imaging modalities of the first imaging source 13101 and the second imaging source 13102 may be the CT imaging modality, and the imaging beams emitted by the first imaging source 13101 may be projected onto two joined detectors of the four detectors 13200 (e.g., the two joined detectors disposed on a side of the preset axis opposite to the first imaging source 13101). The imaging beams emitted by the second imaging source 13102 may be projected onto other two joined detectors of the four detectors 13200 (e.g., the two joined detectors disposed on a side of the preset axis opposite to the second imaging source 13102).

In FIG. 21, both the imaging modalities of the first imaging source 13101 and the second imaging source 13102 may be the DR imaging modality, and the imaging beams emitted by the first imaging source 13101 may be projected onto one of two joined detectors of the four detectors 13200 (e.g., the two joined detectors disposed on a side of the preset axis opposite to the first imaging source 13101) that is far away from the second imaging source 13102. The imaging beams emitted by the second imaging source 13102 may be projected onto one of other two joined detectors of the four detectors 13200 (e.g., the two joined detectors disposed on a side of the preset axis opposite to the second imaging source 13102) that is away from the first imaging source 13101.

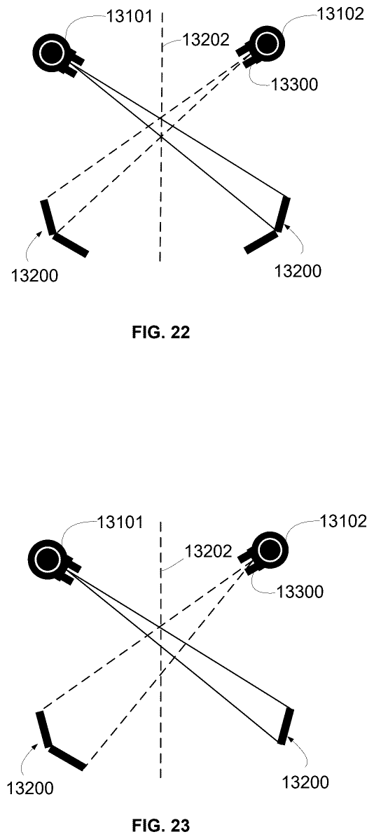

In FIG. 22, both the imaging modalities of the first imaging source 13101 and the second imaging source 13102 may be the DR imaging modality, and the imaging beams emitted by the first imaging source 13101 may be projected onto one of two joined detectors of the four detectors 13200 (e.g., the two joined detectors disposed on a side of the preset axis opposite to the first imaging source 13101) that is close to the second imaging source 13102. The imaging beams emitted by the second imaging source 13102 may be projected onto one of other two joined detectors of the four detectors 13200 (e.g., the two joined detectors disposed on a side of the preset axis opposite to the second imaging source 13102) that is close to the first imaging source 13101.

In some embodiments, the imaging device 1310 may include three detectors 13200, the first imaging source 13101 may emit imaging beams corresponding to the DR imaging modality, and the second imaging source 13102 may switchably emit imaging beams corresponding to the CT imaging modality and imaging beams corresponding to the DR imaging modality. In FIG. 23, the imaging beams emitted by the first imaging source 13101 may be projected onto one of the three detectors 13200 disposed on a side of the preset axis opposite to the first imaging source 13101, and the imaging beams of the CT imaging modality emitted by the second imaging source 13102 may be projected onto two joined detectors of the three detectors 13200 disposed on a side of the preset axis opposite to the second imaging source 13102.

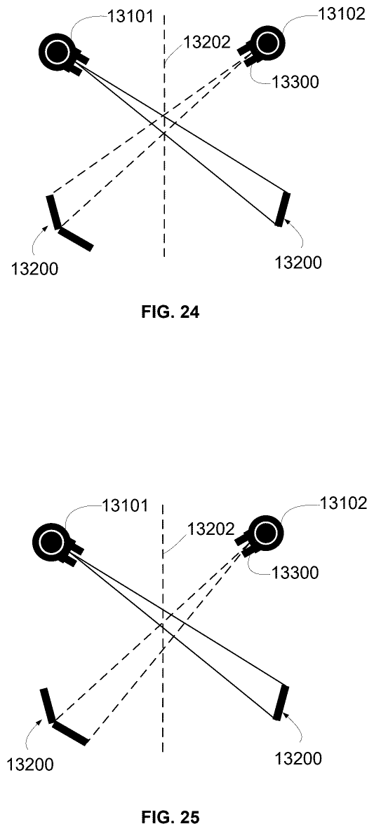

In FIG. 24, the imaging beams emitted by the first imaging source 13101 may be projected onto one of the three detectors 13200, and the imaging beams of the DR imaging modality emitted by the second imaging source 13102 may be projected onto one of two joined detectors of the three detectors (e.g., the two joined detectors disposed on a side of the preset axis opposite to the second imaging source 13102) that is close to the first imaging source 13101.

In FIG. 25, the imaging beams emitted by the first imaging source 13101 may be projected onto one of the three detectors 13200, and the imaging beams of the DR imaging modality emitted by the second imaging source 13102 may be projected onto one of two joined detectors of the three detectors 13200 (e.g., the two joined detectors disposed on a side of the preset axis opposite to the second imaging source 13102) that is away from the first imaging source 13101.

In some embodiments, the imaging device 1310 may further include a gantry. The first imaging source 13101, and the second imaging source 13102, and/or the plurality of detectors 13200 may be disposed on the gantry (not shown). The gantry may be rotatable. For example, the gantry may be circular or curved, which is not limited herein. By controlling the rotation of the gantry, setting (spatial) positions of the first imaging source 13101, the second imaging source 13102, and the plurality of detectors 13200 may be changeable, so that the setting mode of the first imaging source 13101 and the second imaging source 13102 in the imaging device 1310 may be changed from the setting mode shown in FIGS. 15-19 to the setting mode shown in FIGS. 20-25. In some embodiments, the detector(s) 13200 may be arranged along a circumferential direction of the gantry (as shown in FIGS. 13-31). In some embodiments, an included angle of two adjacent detectors of the plurality of detectors may be in a range from 90° to 180° (e.g., 120°, 150°, 180°, or ang angle from 120° to) 180°. Alternatively, or additionally, two adjacent segments of a detector may be in a range from 90° to 180° (e.g., 120°, 150°, 180°, or ang angle from 120° to 180°). In some embodiments, included angles of different adjacent detectors may be the same or different. In some embodiments, the imaging source(s) 13100 may be arranged along a circumferential direction of the gantry (as shown in FIGS. 13-31).

In some embodiments, a plurality of setting modes of the first imaging source 13101 and the second imaging source 13102 may be provided in the case where the one or more imaging sources 13100 at least include one first imaging source 13101 and one second imaging source 13102, so that a user may set according to the setting modes, thereby improving the practicability of the imaging device 1310.

In some embodiments, the one or more imaging sources 13100 may include one first imaging source 13101 and at least two second imaging sources 13102. The at least two second imaging sources 13102 may be respectively disposed on two sides of an axis of the first imaging source 13101.

The first imaging source 13101 and the second imaging source 13102 may be two imaging sources that each emit a specified type of imaging beams, and the specified types of imaging beams emitted by the two imaging sources may be different (e.g., the first imaging source 13101 may be a CT imaging source and the second imaging source 13102 may be a DR imaging source). In some embodiments, each of the two imaging sources can emit at least two different types of imaging beams (e.g., each of the first imaging source 13101 and the second imaging source 13102 may switchably emit CT imaging beams and DR imaging beams). In some embodiments, one of the two imaging sources may emit a specified type of imaging beams, and the other one of the two imaging sources may switchably emit at least two different types of imaging beams (e.g., the first imaging source 13101 may be a DR imaging source, and the second imaging source 13102 may switchably emit CT imaging beams and DR imaging beams). The plurality of second imaging sources 13102 may be the same or different, which is not limited in the embodiments as long as the functions are realized.

In some embodiments, the first imaging source 13101 and the second imaging source 13102 may emit the imaging beams simultaneously, or emit the imaging beams in a staggered manner, or emit the imaging beams sequentially.

In some embodiments, the axis of the first imaging source 13101 may refer to the axis of symmetry 13201 of the plurality of detectors 13200. In the case where the one or more imaging sources 13100 include one first imaging source 13101 and at least two second imaging sources 13102, the first imaging source 13101 may be disposed at any position on the axis of symmetry 13201 of the plurality of detectors 13200, and the at least two second imaging sources 13102 may be disposed on two sides of the axis of the first imaging source 13101. If the axis of the first imaging source 13101 is in a horizontal direction, at least one second imaging source 13102 may be disposed above the axis of the first imaging source 13101, and at least one other second imaging source 13102 may be disposed below the axis of the first imaging source 13101.

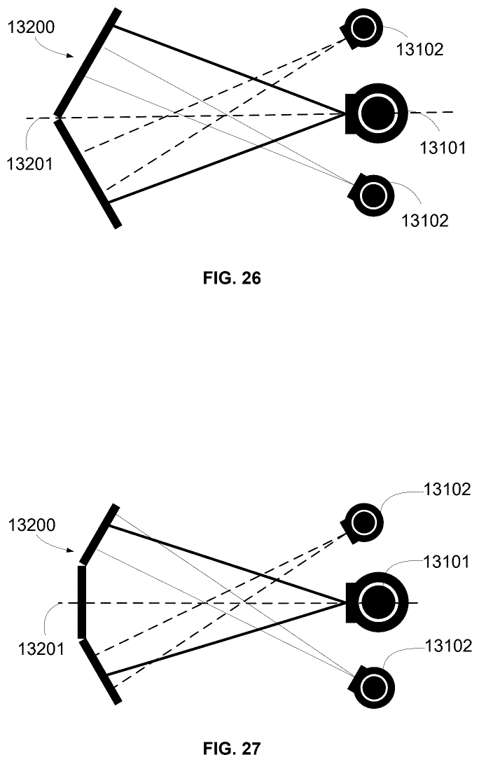

In some embodiments, when the one or more imaging sources 13100 include two second imaging sources 13102, as shown in FIGS. 26-28, the two second imaging sources 13102 may be symmetrically disposed on two sides of the axis of the first imaging source 13101. The imaging modality of the imaging source 13101 may be a CT imaging modality, and the imaging modality of the second imaging source 13102 may be a DR imaging modality. In FIG. 26, the imaging device 1310 may include two detectors 13200, the imaging beams emitted by the first imaging source 13101 may be projected onto the two detectors 13200, and the imaging beams emitted by the second imaging source 13102 may be projected onto one of the two detectors 13200 that is far away from the second imaging source 13102.

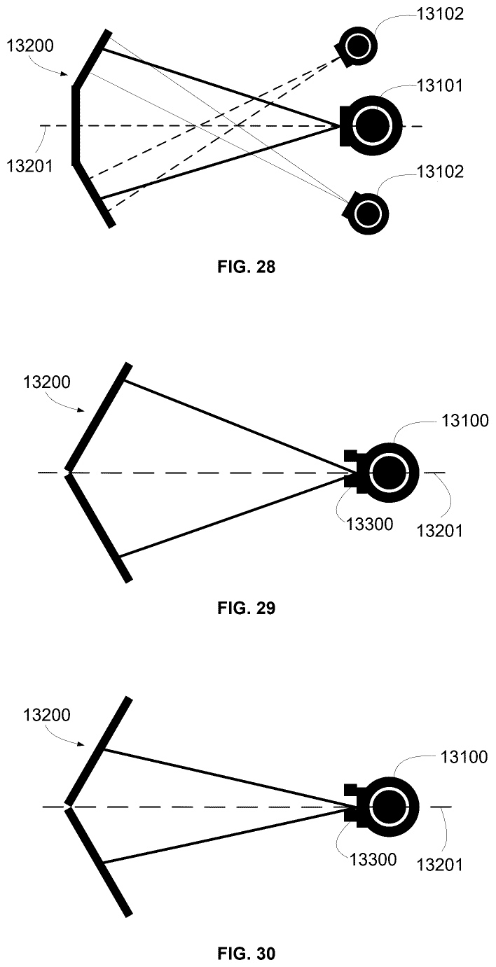

In FIGS. 27-28, the imaging device 1310 may include three detectors 13200, the imaging beams emitted by the first imaging source 13101 may be projected onto the three detectors 13200, and the imaging beams emitted by the second imaging source 13102 may be projected onto one of the three detectors 13200 that is far away from the second imaging source 13102.

In some embodiments, a setting mode of the first imaging source 13101 and the second imaging source 13102 may be provided in the case where the one or more imaging sources 13100 include one first imaging source 13101 and at least two second imaging sources 13102, so that the user may set according to the setting mode, thereby improving the practicability of the imaging device 1310.

In some embodiments, the one or more imaging sources 13100 may include one imaging source 13100. The one imaging source 13100 may be disposed on the axis of symmetry 13201 of the joined structure of the plurality of detectors 13200. The one imaging source 13100 may be configured to emit the at least two different types of imaging beams. For example, the one imaging source 13100 may switch between different imaging modalities as required.

As shown in FIGS. 29-30, the imaging device 1310 may include one imaging source 13100. The one imaging source 13100 may switch between the CT imaging modality and the DR imaging modality. FIG. 29 shows that the imaging modality of the imaging source 13100 is the CT imaging modality, and the imaging beams emitted by the imaging source 13100 are fan-shaped. FIG. 30 shows that the imaging modality of the imaging source 13100 is the DR imaging modality, and the imaging beams emitted by the imaging source 13100 are cone-shaped.

In the embodiment, a setting mode of the imaging source 13100 and a working mode of the imaging source 13100 may be provided in the case where the imaging device 1310 includes the one imaging source 13100, so that the user may select the setting mode according to the actual application, thereby improving the practicality of the imaging device 1310. Moreover, the at least two different types of imaging beams may be emitted using one imaging source 13100, so that the space occupied by the imaging device 1310 may be reduced, and the imaging device 1310 may have higher practicability.

In some embodiments, at least one of the plurality of detectors 13200 may be configured to receive at least two different types of imaging beams. The imaging device 1310 may include the plurality of detectors 13200. The at least one of the detectors 13200 may be configured to receive the at least two different types of imaging beams emitted by the one or more imaging sources 13100. That is, the at least one detector 13200 may be shared by the one or more imaging sources 13100 having different imaging modalities.

In some embodiments, the plurality of detectors 13200 may include a first detector and a second detector. A count of the first detectors may be one or more. Similarly, a count of the second detectors may be one or more. The first detector refers to a detector that receives at least two different types of imaging beams, and the second detector refers to a detector that receives only one specified type of imaging beams. At least one of the plurality of detectors 13200 may be configured to receive at least two different types of imaging beams, and thus, the detectors 13200 may be utilized efficiently and a count of disposed detectors 13200 may be reduced, the space occupied by the imaging device 1310 may be reduced, and the imaging device 1310 may have higher practicability.

As shown in FIG. 15, the imaging device 1310 may further include at least one beam limiter 13300. Each of the at least one beam limiter 13300 may be configured to adjust the imaging beams emitted by one of the one or more imaging sources 13100 that corresponds to the each of the at least one beam limiter.

The beam limiter 13300 may be disposed on one side of each imaging source included in the imaging device 1310 that emits the imaging beams, that is, each beam limiter 13300 may be disposed on the imaging source that corresponds to the each beam limiter 13300. The beam limiter 13300 may be configured to adjust a fan angle and/or a width of the imaging beams emitted from the imaging source 13100 to realize various imaging modalities of the imaging source 13100.

In some embodiments, the working modality of the imaging source 13100 may be switched by adjusting the beam limiter 13300 coupled to the imaging source 13100, one or more parameters (e.g., energy, current, etc.) of the bulb tube of the imaging source 13100, and one or more parameters (e.g., frame rate of imaging, resolution, gain mode, reading mode, etc.) of the detector 13200 corresponding to the imaging source 13100. In some embodiments, the beam limiter 13300 may be coupled to (e.g., disposed on) the imaging source 13100, and the fan angle and/or width of the imaging beams emitted from the imaging source 13100 may be adjusted, so that the user may conveniently adjust the imaging beams according to the actual application, and the practicality of the imaging device 1310 may be improved.

In some embodiments, at least two detectors of the plurality of detectors 13200 may be detachably joined with each other. Merely by way of example, two adjacent detectors 13200 of the plurality of detectors 13200 included in the imaging device 1310 may be detachably joined with each other. That is, if the imaging device 1310 includes two detectors 13200, the two detectors 13200 may be detachably joined with each other. If the imaging device 1310 includes three detectors 13200, two of the three detectors 13200 may be fixedly joined, and the two detectors that are fixedly joined and the other detector 13200 may be detachably joined. Alternatively, two adjacent detectors 13200 of the three detectors 13200 may be detachably joined with each other. The specific manner in which the plurality of detectors 13200 are detachably joined with each other is not limited in the present disclosure as long as the functions are realized.

Merely by way of example, the manner of detachably joining may include a detachable bolt-nut connection, a mortise-and-tenon connection, a plug-jack connection, a magnetic connection, etc. In some embodiments, a connection component (not shown) may be disposed at a connection end of one or more detectors 13200. The detector(s) 13200 may be detachably joined with other detector(s) through the connection component(s), for example, threaded joining, snap joining, hinged connection, etc.

In some embodiments, at least two of the plurality of detectors 13200 may be detachably joined with each other, so that the user may adjust the count of detectors 13200 according to the actual application requirements, and the imaging device 1310 may have higher practicability and flexibility. The at least two of the plurality of detectors 13200 may be joined in a non-detachable manner, for example, welding, bonding, etc.

In some embodiments, the detectors 13200 may be joined and/or unjoined automatically. For example, the two detectors 13200 may be initially located in two separated positions, the two detectors 13200 may be automatically controlled to move closer to each other according to a command of the user or autonomously, and the two detectors 13200 may be finally joined and combined when leaning together by means of a joining manner such as a lapping, snapping, or magnetic suction, etc. Additionally, or alternatively, the two detectors 13200 may be automatically disengaged from the joining state and become separate from each other when they need to be unjoined.

The imaging source 13100 and/or detector 13200 of the imaging device 1310 may be moved to change position(s). The switching between different states of the imaging device 1310 may be achieved by changing the position of the imaging source(s) 13100 and/or the detector(s) 13200 in conjunction with joining/unjoining of the detectors 13200. As an illustrative example, a first state of the imaging device 1310 is shown in FIG. 23. Through the movement of one or two of the two imaging sources 13100 and the corresponding detector(s) 13200, the imaging device 1310 may become a second state shown in FIG. 17, and the detectors 13200 may be joined together to form a detection module combined by three detectors 13200 for imaging. In addition, the state switching of the imaging device 1310 may be determined as required. For example, the imaging device 1310 may be switched between different states shown in FIGS. 17 and 25, and switched between different states shown in FIGS. 14 and 17 (e.g., by activating/deactivating or moving in/out the corresponding detector(s) 13200).

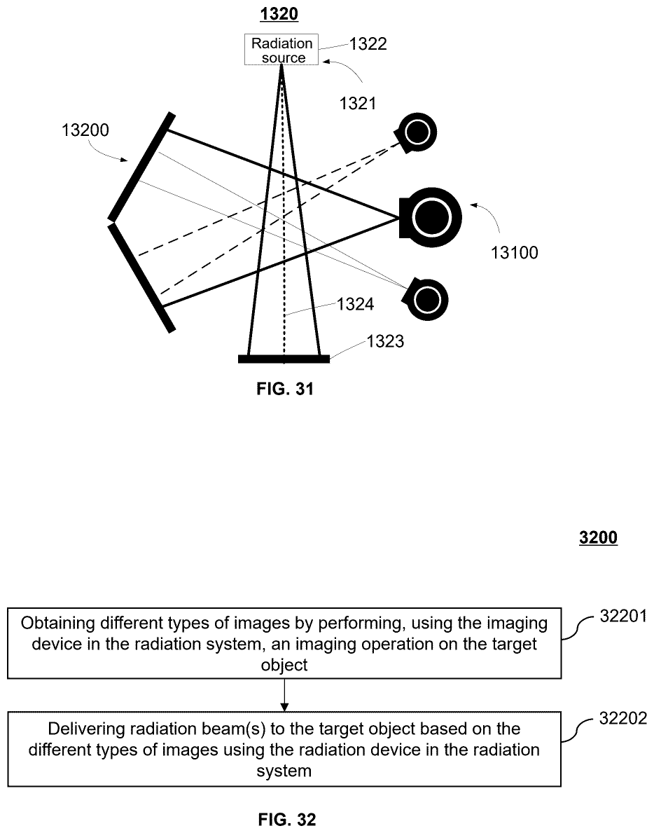

As shown in FIG. 31, one embodiment of the present disclosure provides the radiation system 1320. The radiation system 1320 may include the imaging device 1310 and the radiation device 1321 configured to deliver a radiation beam (also referred to as a treatment beam) to a target object based on an image obtained using the imaging device 1310.

The imaging beams may be emitted through the imaging source(s) 13100 in the imaging device 1310. The imaging beams may be projected through the target object onto the corresponding detector(s) 13200, and the image may be obtained. The radiation device 1321 may determine, based on the image obtained using the imaging device 1310, a target region in the target object, and deliver the radiation beam to the target object.

The radiation system 1320 provided in the embodiments of the present disclosure may include the imaging device 1310 provided in the embodiments, so that the radiation system 1320 may have all the beneficial effects of the imaging device 1310, which may not be repeated herein. In addition, the radiation system 1320 may deliver the radiation beam to the target object more accurately based on the image(s) obtained using the imaging device 1310, thereby improving the practicality and reliability of the radiation system 1320.

As shown in FIG. 31, in some embodiments, the radiation device 1321 may include a radiation source 1322 (also referred to as a treatment head) and a detection component 1323. The radiation source 1322 and the detection component 1323 may be disposed relative to each other.

The radiation source 1322 may be configured to deliver the radiation beam, and the radiation beam may be projected through the target object onto the detection component 1323. For example, the detection component 1323 may generate an image and/or determine a detection value of radiation dose by analyzing received signals. The radiation device 1321 may determine, based on the image and/or the detection value of radiation dose, an actual radiation dose distribution of the radiation beam in the target object, thereby obtaining a difference between an actual radiation dose distribution and a planned radiation dose distribution, and may further determine whether the radiation beam delivered by the radiation source 1322 conforms to a planned radiation beam specified by a preset treatment plan. The detection component 1323 may be or include an Electronic Portal Imaging Device (EPID). A type and structure, etc., of the radiation source 1322 and the detection component 1323 is not limited in the embodiment as long as the functions thereof are realized.

A connection line along which the radiation beam is delivered by the radiation source 1322 (e.g., a line from a center in the radiation source 1322 to a center of the detection component 1323) may be a preset connection line 1324. In some embodiments, each imaging source 13100 in the imaging device 1310 and the corresponding detector 13200 may be disposed on two sides of the preset connection line 1324.

An imaging method is provided. The method may be applied to the imaging device provided in the above embodiments, and the method may include obtaining different types of images by performing an imaging operation on a target object using the imaging device. The imaging beams emitted by the imaging source(s) in the imaging device may be projected through the target object onto the detector(s), and image(s) may be obtained by analyzing and processing the signal(s) received by the detector(s). The imaging source(s) in the imaging device may emit at least two different types of imaging beams, so that the different types of images may be obtained using the imaging device.

The imaging method provided in some embodiments may be realized using the imaging device, so that the imaging method may have all the beneficial effects of the imaging device, which may not be repeated herein. In some embodiments, as shown in FIG. 32, a radiation method is provided. The method may be applied to the radiation system provided in the above embodiment, and the method may include one or more of the following operations:

In 32201, different types of images may be obtained by performing, using the imaging device in the radiation system, an imaging operation on the target object. More descriptions regarding the performing an imaging operation on the target object using the imaging device may be found in the descriptions of the above embodiments, which may not be repeated herein.

In 32202, the radiation beam may be delivered to the target object based on the different types of images using the radiation device in the radiation system. After obtaining the different types of images determined by the imaging device, the radiation system may determine, based on the images, a target region in the target object, and deliver, based on a position of the determined target region and critical organs around the target region, the radiation beam to the target object.

The radiation method provided in the embodiment is applied to the radiation system provided in the above embodiments, so that the radiation method may have all the beneficial effects of the radiation system, which may not be repeated herein.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A non-transitory computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. An imaging device, comprising:
one or more imaging sources; and
a plurality of detectors combined by joining, the plurality of detectors being configured to receive one or more imaging beams emitted by the one or more imaging sources.

2. The imaging device of claim 1, wherein the plurality of detectors are joined to form a detection surface, and at least a portion of the detection surface is bent or curved.

3. The imaging device of claim 1, wherein:
the one or more imaging sources include a first imaging source and a second imaging source, and
the second imaging source is disposed on either side of an axis of the first imaging source, or the first imaging source and the second imaging source are respectively disposed on two sides of a preset axis.

4. The imaging device of claim 1, wherein the one or more imaging sources include a first imaging source and at least two second imaging sources, the at least two second imaging sources being respectively disposed on two sides of an axis of the first imaging source.

5. The imaging device of claim 1, wherein the one or more imaging sources include one imaging source, the one imaging source being disposed on an axis of symmetry of a joined structure of the plurality of detectors, and the one imaging source being configured to emit the one or more imaging beams.

6. The imaging device of claim 1, wherein at least one detector of the plurality of detectors is configured to receive the one or more imaging beams.

7. The imaging device of claim 1, further comprising at least one beam limiter, wherein each of the at least one beam limiter is configured to adjust an imaging beam emitted by an imaging source of the one or more imaging sources that corresponds to the each of the at least one beam limiter.

8. The imaging device of claim 1, wherein at least two of the plurality of detectors are detachably joined with each other.

9. The imaging device of claim 1, further comprising a gantry, wherein:
the plurality of detectors are disposed on the gantry and are arranged along a circumferential direction of the gantry; and/or
one or more imaging sources are disposed on the gantry and are arranged along the circumferential direction of the gantry.

10. The imaging device of claim 1, wherein an included angle of two adjacent detectors of the plurality of detectors is in a range from 120° to 180°.

11. The imaging device of claim 1, wherein plurality of detectors include one or more detectors with a relatively large area and one or more detectors with a relatively small area.

12. An imaging device, comprising:
one or more imaging sources configured to emit at least two different types of imaging beams; and
one or more detectors configured to receive the at least two different types of imaging beams to generate images of at least two different modalities or with at least two different energy levels.

13. The imaging device of claim 12, wherein the one or more imaging sources include one imaging source, and wherein:
the one imaging source is configured to be switched between at least two imaging modalities, each of the at least two imaging modalities corresponding to emission of one type of the at least two different types of imaging beams.

14. The imaging device of claim 12, wherein the one or more imaging sources include two or more imaging sources, and wherein:
each of the two or more imaging sources is configured to emit a corresponding one type of the at least two different types of imaging beams.

15. The imaging device of claim 12, wherein the one or more imaging sources include two or more imaging sources, and wherein:
each of the two or more imaging sources is configured to be switched between at least two imaging modalities, each of the at least two imaging modalities corresponding to emission of one type of the at least two different types of imaging beams, such that the two or more imaging sources emit the at least two different types of imaging beams.

16. The imaging device of claim 12, wherein the one or more imaging sources include two or more imaging sources, and wherein:
the two or more imaging sources are configured to emit the at least two different types of imaging beams simultaneously, alternately, or asynchronously.

17. The imaging device of claim 12, wherein the one or more imaging sources include two or more imaging sources, and wherein:
the two or more imaging sources include a first type of imaging source and a second type of imaging source, a first axis of the first type of imaging source and a second axis of the second type of imaging source have a preset angle; or
the two or more imaging sources include a first type of imaging source and at least two second type of imaging sources, the at least two second type of imaging sources being respectively disposed on two sides of the first axis of the first type of imaging source.

18. The imaging device of claim 12, wherein:
the one or more detectors include one detector; or
the one or more detectors include two or more detectors combined by joining; or
the one or more detectors include at least two sets of detectors that are respectively disposed on two sides of a preset axis.

19. The imaging device of claim 12, wherein:
the one or more imaging sources include a computed tomography (CT) imaging source and at least one digital radiography (DR) imaging source; or
the one or more imaging sources include two DR imaging sources.

20. A radiation system, comprising:
an imaging device configured to scan a target object to generate one or more images of the target object; and
a radiation device configured to deliver a radiation beam to the target object based on the one or more images;
wherein the imaging device includes:
one or more imaging sources; and
a plurality of detectors combined by joining, the plurality of detectors being configured to receive one or more imaging beams emitted by the one or more imaging sources.
